(19) United States
(12) Patent Application Publication
(10) Pub. No.: US 2004/0072992 A1
(43) Pub. Date: Apr. 15, 2004

(54) Novel peptide capable of specifically acting on biological membrane

(76) Inventors: Sachiko Machida, Tsukuba-shi (JP); Ken Tokuyasu, Tsukuba-shi (JP); Shigeru Matsunaga, Ryugasaki-shi (JP); Yoshikiyo Sakakibara, Tsukuba-shi (JP); Masuko Kobori, Ushiku-shi (JP); Zhesheng Wen, GuangZhou (CN)

Correspondence Address: MINTZ LEVIN COHN FERRIS GLOVSKY & POPEO, 666 THIRD AVENUE, NEW YORK, NY, 10017, US

(21) Appl. No.: 10/651,563

(22) Filed: Aug. 29, 2003

(30) Foreign Application Priority Data
Aug. 30, 2002 (JP) 2002-253169
Jan. 29, 2003 (JP) 2003-021198

(51) Int. Cl. C07K 7/08

(52) U.S. Cl. 530/327

(57) ABSTRACT

A peptide is provided, consisting of amino acid sequence comprising an amino acid sequence Z¹X¹X²X³Z²X⁴X⁵Z³X⁶X⁷X⁸Z⁴X⁹. where X¹-X⁹ are any amino acids and at least two amino acids of Z¹-Z⁴ are basic amino acids. An analog thereof is also provided.

### BACKGROUND OF THE INVENTION

1. Field of the Invention

The present invention relates to a novel peptide capable of specifically acting on biological membranes, and a method for screening for the peptide. More particularly, the present invention relates to a peptide which specifically acts on the membrane of microorganisms and does not act on normal animal cell membranes, and a method for screening for the peptide. The present invention also relates to a peptide capable of specifically forming pores in the membrane of microorganisms and incapable of hemolysis, and a method for screening for the peptide. The present invention also relates to an antibacterial peptide for killing microorganisms. Specifically, the present invention relates to an antibacterial peptide for microorganisms causing the putrefaction of food or industrial products. The present invention also relates to an antibacterial peptide for microorganisms capable of infecting animals and/or plants. The present invention also relates to a pharmaceutical composition for killing microorganisms, a pharmaceutical composition for treating infectious diseases caused by microorganisms, a pharmaceutical composition for treating cancer, or a pharmaceutical composition for suppressing apoptosis, which comprise the above-described peptide.

2. Description of the Related Art

There are naturally occurring peptides capable of acting on biological membranes and forming pores therein (pore forming ability). These peptides are called membrane perturbation peptides, and are extensively present in plants, insects, invertebrate animals, and vertebrate animals including humans. It is believed that the peptides specifically recognize and act on the biological membranes of a wide range of organisms including microorganisms. Particularly, a certain membrane perturbation peptide, which acts on the biological membrane of microorganisms, kills microorganisms by destroying the barrier function of their biological membranes. Such a isolated peptide is reviewed in, for example, Michael Zasloff, Nature 415:389-395 (2002) and Katsumi Matsuzaki, “Kokinsei Seitai Bogyo Peputido: Sayo Kiko to sono Oyo [Antibacterial Biophylactic Peptide: Mechanism and its Application], FFI JOURNAL, No.190:23-27 (2001), and is disclosed in, for example, Japanese Laid-Open Publication No. 2001-288105, Japanese Laid-Open Publication No. 2001-186887, Japanese Laid-Open Publication No. 2001-2582, Japanese Laid-Open Publication No. 2000-63400, Japanese National phase PCT Laid-Open Publication No. 2002-503701 (WO99/42119), Japanese National phase PCT Laid-Open Publication No. 2002-503641 (WO99/26971), Japanese National phase PCT Laid-Open Publication No. 2001-527412 (W098/09796), Japanese National phase PCT Laid-Open Publication No. 2001-517422 (WO99/15548), Japanese National phase PCT Laid-Open Publication No. 9-512711 (WO95/30751), and the like.

Examples of membrane perturbation peptides for microorganisms include honeybee toxin-derived melittin, hornet toxin-derived mastoparanX and Xenopus laevis-derived magainin 2. Melittin and mastoparanX act on the membrane of microorganisms as well as exhibit the hemolytic activity. Magainin 2 does not exhibit the hemolytic activity and has the specific pore forming activity in the membrane of microorganisms. Mast 21, which is a peptide obtained by modifying the backbone of mastoparanX, has the specific pore forming activity in the membrane of microorganisms and lacks the hemolytic activity (see, Japanese Patent No. 2967925). Peptides, which do not exhibit the hemolytic activity and have the capability to specifically forming pores in the membrane of microorganisms, are called host defense peptides. The host defense peptide of the present invention serves as an antibacterial peptide for killing microorganisms which cause the putrefaction of food or industrial products, or an antibacterial peptide for killing microorganisms which infect animals and/or plants. In addition, the peptide of the present invention may be used as an antibiotic having a low potential for producing antibiotic-resistant microbial species.

Since host defense peptides specifically target the membrane of microorganisms, they may be used as a signal for a drug delivery system which is a mechanism for delivering a desired drug to foreign matter, such as a microorganism or the like, which invades the body. Therefore, the peptide of the present invention is expected to be used in a variety of applications. For example, the peptide of the present invention is used as a signal for a drug delivery system in which a required amount of drug is applied only to a required site in the body so that the effect of the drug is maximally elicited.

The biological membrane of microorganisms is distinguished from the biological membrane of animal cells in the structure relating to phospholipids contained in the membrane. Whereas acid phospholipids, such as phosphatidyl glycerol, phosphatidyl serine, cardiolipin, and the like, are present on the surface of the membrane of microorganisms, no acid phospholipids are exposed on the surface of the membrane of healthy animal cells and, conversely, abundant cholesterols are present, which are not present on the membrane of microorganisms. Host defense peptides are believed to recognize the difference in the membrane structure and form pores in microbial membranes. However, the antibacterial property of each known host defense peptide is not effective for all bacteria, fungi, and the like, but shows different antibacterial spectra. In addition, it has been reported that host defense peptides are not stable. Thus, host defense peptides are not practically used as antibacterial peptides. An attempt has been made to modify a peptide having a known sequence to improve an activity for a predetermined purpose, for example (see, e.g., Sachiko Machida et al., Biosci. Biotechnol. Biochem., 64:985-994 (2000); and Song Yub Shin et al, Biochemical Biophysical Research Communications, 275:904-909 (2000)). However, there has been no technique for screening random libraries for a peptide having a particular amino acid sequence which can recognize the structure of cell membranes and act on the cell membranes.

To obtain a host defense peptide having a stable and/or desired antibacterial spectrum, an attempt has been made to modify a known peptide sequence to produce a peptide having an improved activity for a predetermined purpose. For example, in recent years, high throughput screening has been frequently carried out using combinatorial chemistry or the like (see, e.g., Sylvie E. Blondelle and Richard A. Houghten, TIBTECH, 14:60-64 (1996); Hong, S. Y. et al., Antimicrobial Agents and Chemotherapy, 42:2534-2541 (1998); and Kyoung-Chul Choi et al., Biotechnology Letters, 24:251-256 (2002)). However, no successful example has been reported in which a peptide capable of specifically acting on the membrane of microorganisms was obtained from random peptide libraries. In addition, it is difficult both to synthesize peptide libraries having a certain chain length or more and to directly determine the amino acid sequence of a peptide after screening. To avoid this, by associating the amino acid sequence of a peptide with genetic information, the amino acid sequence of a peptide has been determined from a base sequence encoding the peptide (e.g., phage display, bacterial surface display, yeast surface display, etc. (e.g., Nobuhide Tsuchiya and Hiroshi Yanagawa, “Shinka Bunshi Kogaku wo Kosei suru Gijyutsu (3) Idensikata to Hyogenkei no Taiokagijyutsu [Evolutionary Molecular Engineering (3) Techniques for Associating Genotype and Phenotype”, Kagaku to Seibutsu [Chemistry and Biology], 37:811-815 (1999), and N. Doi and H. Yanagawa, Combinatorial Chemistry & High Throughput Screening, 4:497-509 (2001)). However, all of these techniques use living cells for synthesis of proteins, including a step of utilizing the function of an organism, such as a microorganism, a phage, or the like. Therefore, it was not possible to use these techniques to select the amino acid sequence of a peptide having a potential to be lethal to organisms or affect the growth of organisms, such as a host defense peptide.

In contrast, an in vitro technique, such as a ribosome display method, an emulsion method, and the like, has been developed, in which all processes in screening are carried out in vitro in a cell-free system, i.e., without utilizing the function of a living cell, such as a microorganisms, a phage, or the like (e.g., N. Doi and H. Yanagawa, Combinatorial Chemistry & High Throughput Screening, 4:497-509 (2001), Anthony D. Keefeand Jack W. Szostak, Nature, 410(2001), and Patrick Amstutz et al., Current Opinion in Biotechnology, 406-405(2001)). Particularly, in the case of ribosome display, a protein-ribosome-mRNA complex (PRM complex) is formed and a protein having a particular function is screened for. With such a method, it is possible to select the amino acid sequence of a peptide having a potential to be lethal to organisms or affect the growth of organisms.

However, it is difficult to develop selection pressure in screening proteins. At present, the above-described methods have been developed mainly for methodology. Therefore, no successful method for screening for a novel peptide capable of specifically acting on biological membranes has been reported.

### SUMMARY OF THE INVENTION

According to an aspect of the present invention, a peptide is provided, consisting of amino acid sequence comprising an amino acid sequence Z¹X¹X²X³Z²X⁴X⁵Z³X⁶X⁷X⁸Z⁴X⁹, wherein X′—X⁹ are any amino acids and at least two amino acids of Z¹-Z⁴ are basic amino acids. An analog thereof may be provided.

In one embodiment of this invention, the basic amino acids are lysine (K) or arginine (R).

In one embodiment of this invention, at least two amino acids of the X¹-X⁹ are hydrophobic amino acids.

In one embodiment of this invention, the peptide comprises a sequence selected from the group consisting of ALR, WALR, WGALR, RLAWG, GWALR, RVL, KVL, RVG, KVG, GVR, VGR, RVA, RSV, RVS, KVS, SVK, and VSK. An analog thereof may be provided.

In one embodiment of this invention, the peptide comprises a sequence selected from the group consisting of ALR, RLAWG, GWALR, RVL, KVL, RVG, andKVG. An analog thereof may be provided.

In one embodiment of this invention, the peptide comprises a sequence selected from the group consisting of ALR, KVL, and RVG. An analog thereof may be provided.

According to another aspect of the present invention, a peptide capable of specifically acting on a membrane of a microorganism is provided, comprising a sequence selected from the group consisting of ALR, WALR, WGALR, RLAWG, GWALR, RVL, KVL, RVG, KVG, GVR, VGR, RVA, RSV, RVS, KVS, SVK, and VSK. An analog thereof may be provided.

In one embodiment of this invention, the microorganism causes putrefaction of food or industrial products.

In one embodiment of this invention, the peptide comprises a sequence selected from the group consisting of ALR, RLAWG, GWALR, RVL, KVL, RVG, and KVG. An analog thereof may be provided.

In one embodiment of this invention, the peptide comprises a sequence selected from the group consisting of ALR, KVL, and RVG. An analog thereof may be provided.

According to another aspect of the present invention, a peptide capable of specifically acting on a membrane of an animal cell having an abnormality is provided, comprising a sequence selected from the group consisting of ALR, WALR, WGALR, RLAWG, GWALR, RVL, KVL, RVG, KVG, GVR, VGR, RVA, RSV, RVS, KVS, SVK, and VSK. An analog thereof may be provided.

In one embodiment of this invention, the animal cell having an abnormality is a cancer cell.

In one embodiment of this invention, the peptide comprises a sequence selected from the group consisting of ALR, RLAWG, GWALR, RVL, KVL, RVG, and KVG. An analog thereof may be provided.

In one embodiment of this invention, the peptide comprises a sequence selected from the group consisting of ALR, KVL, and RVG. An analog thereof may be provided.

In one embodiment of this invention, the animal cell having an abnormality is an apoptotic cell.

In one embodiment of this invention, the peptide comprises a sequence selected from the group consisting of ALR, RLAWG, GWALR, RVL, KVL, RVG, and KVG. An analog thereof may be provided.

In one embodiment of this invention, the peptide comprises a sequence selected from the group consisting of ALR, KVL, and RVG. An analog thereof may be provided.

According to another aspect of the present invention, a peptide is provided, comprising: i) a first sequence selected from the group consisting of ALR, WALR, WGALR, RLAWG, GWALR, RVL, KVL, RVG, KVG, GVR, VGR, RVA, RSV, RVS, KVS, SVK, and VSK; ii) a second sequence selected from the group consisting of ALR, WALR, WGALR, RLAWG, GWALR, RVL, KVL, RVG, KVG, GVR, VGR, RVA, RSV, RVS, KVS, SVK, and VSK; and iii) a third sequence selected from the group consisting of ALR, WALR, WGALR, RLAWG, GWALR, RVL, KVL, RVG, KVG, GVR, VGR, RVA, RSV, RVS, KVS, SVK, and VSK.

According to another aspect of the present invention, a peptide is provided, comprising a repeat of at least three sequences of KVL or ALR.

According to another aspect of the present invention, a peptide is provided, comprising an amino acid sequence selected from the group consisting of SEQ ID NO: 11 to SEQ ID NO: 122. An analog thereof may be provided.

According to another aspect of the present invention, a peptide capable of specifically acting on a membrane of a microorganism is provided, comprising an amino acid sequence selected from the group consisting of SEQ ID NO: 11 to SEQ ID NO: 122. An analog thereof may be provided.

In one embodiment of this invention, the microorganism causes putrefaction of food or industrial products.

According to another aspect of the present invention, a peptide capable of specifically acting on a membrane of an animal cell having an abnormality is provided, comprising an amino acid sequence selected from the group consisting of SEQ ID NO: 11 to SEQ ID NO: 122. An analog thereof may be provided.

In one embodiment of this invention, the animal cell having an abnormality is a cancer cell.

In one embodiment of this invention, the animal cell having an abnormality is an apoptotic cell.

According to another aspect of the present invention, a peptide is provided, comprising an amino acid sequence selected from the group consisting of SEQ ID NO: 11 to SEQ ID NO: 122. A variant thereof is provided. The variant has at least one amino acid deletion, addition, and/or substitution in the amino acid sequence, and maintains a property of specifically acting on a membrane of a microorganism.

In one embodiment of this invention, the microorganism causes putrefaction of food or industrial products.

According to another aspect of the present invention, a peptide is provided, comprising an amino acid sequence selected from the group consisting of SEQ ID NO: 11 to SEQ ID NO: 122. A variant thereof is provided. The variant has at least one amino acid deletion, addition, and/or substitution in the amino acid sequence, and maintains a property of specifically acting on a membrane of an animal cell having an abnormality.

In one embodiment of this invention, the animal cell having an abnormality is a cancer cell.

In one embodiment of this invention, the animal cell having an abnormality is an apoptotic cell.

According to another aspect of the present invention, a peptide is provided, comprising an amino acid sequence selected from the group consisting of SEQ ID NO: 11 to SEQ ID NO: 122. A variant thereof is provided. The variant has at least one conservative amino acid substitution in the amino acid sequence, and maintains a property of specifically acting on a membrane of a microorganism.

In one embodiment of this invention, the microorganism causes putrefaction of food or industrial products.

According to another aspect of the present invention, a peptide is provided, comprising an amino acid sequence selected from the group consisting of SEQ ID NO: 11 to SEQ ID NO: 122. A variant thereof is provided. The variant has at least one conservative amino acid substitution in the amino acid sequence, and maintains a property of specifically acting on a membrane of an animal cell having an abnormality.

In one embodiment of this invention, the animal cell having an abnormality is a cancer cell.

In one embodiment of this invention, the animal cell having an abnormality is an apoptotic cell.

According to another aspect of the present invention, a peptide is provided, comprising a sequence RNWRGIAGMARRLLGRNWRLM. An analog thereof may be provided. The peptide or the analog thereof has an ability to act on a membrane of a microorganism at least two fold higher than a peptide comprising a sequence KNWRGIAGMAKKLLGKNWKLM.

In one embodiment of this invention, the microorganism causes putrefaction of food or industrial products.

According to another aspect of the present invention, a peptide is provided, comprising a sequence RNWRGIAGMARRLLGRNWRLM. An analog thereof may be provided. The peptide or the analog thereof has an ability to act on a membrane of an animal cell having an abnormality at least two fold higher than a peptide comprising a sequence KNWRGIAGMAKKLLGKNWKLM.

In one embodiment of this invention, the animal cell having an abnormality is a cancer cell.

In one embodiment of this invention, the animal cell having an abnormality is an apoptotic cell.

According to another aspect of the present invention, a peptide is provided, comprising a sequence RNWRGIAGMARRLLGRNWRLM. A variant thereof is provided. The variant has at least one amino acid deletion, addition, and/or substitution in the sequence, maintains a property of specifically acting on a membrane of a microorganism, and has an ability to act on a membrane of a microorganism at least two fold higher than a peptide having a sequence KNWRGIAGMAKKLLGKNWKLM.

In one embodiment of this invention, the microorganism causes putrefaction of food or industrial products.

According to another aspect of the present invention, a peptide is provided, comprising a sequence RNWRGIAGMARRLLGRNWRLM. A variant thereof is provided. The variant has at least one amino acid deletion, addition, and/or substitution in the sequence, maintains a property of specifically acting on a membrane of an animal cell having an abnormality, and has an ability to act on a membrane of an animal cell having an abnormality at least two fold higher than a peptide having a sequence KNWRGIAGMAKKLLGKNWKLM.

In one embodiment of this invention, the animal cell having an abnormality is a cancer cell.

In one embodiment of this invention, the animal cell having an abnormality is an apoptotic cell.

According to another aspect of the present invention, a peptide is provided, comprising a sequence RNWRGIAGMARRLLGRNWRLM. A variant thereof is provided. The variant has at least one conservative amino acid substitution excluding K or R in the sequence, maintains a property of specifically acting a membrane of a microorganism, and has an ability to act on a membrane of a microorganism at least two fold higher than a peptide having a sequence KNWRGIAGMAKKLLGKNWKLM.

In one embodiment of this invention, the microorganism causes putrefaction of food or industrial products.

According to another aspect of the present invention, a peptide is provided, comprising a sequence RNWRGIAGMARRLLGRNWRLM. A variant thereof is provided. The variant has at least one conservative substitution excluding K or R in the sequence, maintains a property of specifically acting on a membrane of an animal cell having an abnormality, and has an ability to act on an ability to act on a membrane of an animal cell having an abnormality at least two fold higher than a peptide having a sequence KNWRGIAGMAKKLLGKNWKLM.

In one embodiment of this invention, the animal cell having an abnormality is a cancer cell.

In one embodiment of this invention, the animal cell having an abnormality is an apoptotic cell.

According to another aspect of the present invention, a library is provided, comprising a plurality of nucleic acid sequences, each nucleic acid sequence comprising: (1) a first cassette comprising a base sequence encoding a first peptide; (2) a second cassette comprising a base sequence encoding a second peptide, said base sequence having the same reading frame as that of the base sequence encoding the first peptide, wherein the second peptide comprises a site allowing flexible movement of the first peptide; and (3) a third cassette comprising a base sequence essentially required for transcription and translation of the first and the second cassette, the third cassette being operatively linked to the first and second cassettes. The number of the nucleic acid sequences in the library whose first cassettes are different from one another is at least two.

In one embodiment of this invention, the second cassette further comprises a base sequence encoding a tag sequence.

In one embodiment of this invention, the first cassette does not comprise a termination codon.

In one embodiment of this invention, the number of the nucleic acid sequences whose first cassettes are different from one another is at least 100.

In one embodiment of this invention, the number of the nucleic acid sequences whose first cassettes are different from one another is at least 1000.

According to another aspect of the present invention, a vector is provided, comprising the above-described library.

According to another aspect of the present invention, a method for screening for a nucleic acid encoding a peptide capable of acting on a biological membrane is provided, comprising the steps of: constructing a DNA library; preparing peptides by transcription and translation of DNAs of the library in a cell-free system, and forming complexes of the peptide, a ribosome, and mRNA: and selecting the complex capable of specifically binding to a membrane model.

In one embodiment of this invention, the DNA library comprises the above-described library.

In one embodiment of this invention, the DNA library comprises the above-described library.

In one embodiment of this invention, the membrane model is an artificial lipid bilayer imitating a cell membrane structure of an organism.

In one embodiment of this invention, the membrane model is a membrane model immobilized on a solid phase.

In one embodiment of this invention, the solid phase is a magnetic bead.

In one embodiment of this invention, the method further comprises reverse-transcribing mRNA in the selected complex to DNA.

In one embodiment of this invention, a DNA library is prepared using DNA obtained in the reverse-transcription step, and the complex forming step, the complex selecting step, and the reverse-transcription step are repeated.

In one embodiment of this invention, the number of the repetitions is at least 4.

According to another aspect of the present invention, a pharmaceutical composition for killing a microorganism is provided, comprising the above-described peptide, or an analog or variant thereof.

According to another aspect of the present invention, a pharmaceutical composition for preventing putrefaction of food or industrial products is provided, comprising the above-described peptide, or an analog or variant thereof.

According to another aspect of the present invention, a pharmaceutical composition for killing a microorganism is provided, comprising the above-described peptide, or an analog or variant thereof.

According to another aspect of the present invention, a pharmaceutical composition for preventing putrefaction of food or industrial products is provided, comprising the above-described peptide, or an analog or variant thereof.

According to another aspect of the present invention, a pharmaceutical composition for treating an infectious disease caused by a microorganism is provided, comprising the above-described peptide, or an analog or variant thereof.

According to another aspect of the present invention, a pharmaceutical composition for treating an infectious disease caused by a microorganism is provided, comprising the above-described peptide, or an analog or variant thereof.

According to another aspect of the present invention, an antibiotic is provided, comprising the above-described peptide, or an analog or variant thereof.

According to another aspect of the present invention, a pharmaceutical delivery substance for delivering a drug to a site infected with a microorganism is provided, comprising the above-described peptide, or an analog or variant thereof.

According to another aspect of the present invention, a pharmaceutical delivery substance for delivering a drug to a site infected with a microorganism is provided, comprising the above-described peptide, or an analog or variant thereof.

According to another aspect of the present invention, a pharmaceutical composition for treating a cancer is provided, comprising the above-described peptide, or an analog or variant thereof.

According to another aspect of the present invention, a pharmaceutical composition for treating a cancer is provided, comprising the above-described peptide, or an analog or variant thereof.

According to another aspect of the present invention, a pharmaceutical delivery substance-for delivering a drug to a cancer lesion site is provided, comprising the above-described peptide, or an analog or variant thereof.

According to another aspect of the present invention, a pharmaceutical delivery substance for delivering a drug to a cancer lesion site is provided, comprising the above-described peptide, or an analog or variant thereof.

According to another aspect of the present invention, a pharmaceutical composition for suppressing apoptosis is provided, comprising the above-described peptide, or an analog or variant thereof.

According to another aspect of the present invention, a pharmaceutical composition for suppressing apoptosis is provided, comprising the above-described peptide, or an analog or variant thereof.

According to another aspect of the present invention, a pharmaceutical delivery substance for delivering a drug to a site undergoing apoptosis is provided, comprising the above-described peptide, or an analog or variant thereof.

According to another aspect of the present invention, a pharmaceutical delivery substance for delivering a drug to a site undergoing apoptosis is provided, comprising the above-described peptide, or an analog or variant thereof.

According to another aspect of the present invention, a kit for screening for a nucleic acid encoding a peptide capable of acting on a biological membrane is provided, comprising: a lipid for preparing a membrane model.

In one embodiment of this invention, the kit further comprises an enzyme and ribosome for forming a peptide-ribosome-mRNA complex in a cell-free system.

In one embodiment of this invention, the enzyme and the ribosome are provided as cell free extracts.

In one embodiment of this invention, the cell free extract is S30 extract, i.e., E. coli extract.

In one embodiment of this invention, the kit further comprises the above-described library.

In one embodiment of this invention, the kit further comprises the above-described library.

According to another aspect of the present invention, a pharmaceutical composition for killing a microorganism is provided, comprising a peptide having an amino acid sequence selected from the group consisting of SEQ ID NOs: 5, 107, 121, and 122, or an analog or variant thereof.

According to another aspect of the present invention, a pharmaceutical composition for preventing putrefaction food or industrial products is provided, comprising a peptide having an amino acid sequence selected from the group consisting of SEQ ID NOs: 5, 107, 121, and 122, or an analog or variant thereof.

According to another aspect of the present invention, a pharmaceutical composition for treating an infectious disease caused by a microorganism is provided, comprising a peptide having an amino acid sequence selected from the group consisting of SEQ ID NOs: 5, 107, 121, and 122, or an analog or variant thereof.

In one embodiment of this invention, the microorganism is pathogenic to an animal.

In one embodiment of this invention, the microorganism is pathogenic to a plant.

In one embodiment of this invention, the plant pathogenic microorganism is Erwinia carotovora.

According to another aspect of the present invention, a pharmaceutical composition for treating a cancer is provided, comprising a peptide having an amino acid sequence selected from the group consisting of SEQ ID NOs: 5, 107, 121, and 122, or an analog or variant thereof.

In one embodiment of this invention, the cancer is selected from the group consisting of bladder cancer, stomach cancer, breast cancer, lung cancer, prostate cancer, glioblastoma, large intestine cancer, uterine cancer, ovarian cancer, kidney cancer, and leukemia.

According to the method of the present invention, it is possible to efficiently screen any library for a peptide capable of acting on a biological membrane. Particularly, the method of the present invention can completely remove an influence of a peptide during screening compared to techniques, such as phage display, bacteria surface display, yeast surface display, and the like, in which a living thing, such as a microorganism, a phage, or the like, are used to express a peptide. Therefore, it is possible to produce a peptide lethal to organisms or which affects the growth of organisms.

Thus, the invention described herein makes possible the advantages of providing (1) a method for screening for a novel peptide capable of specifically acting on a biological membrane; and (2) a novel anti-microbial peptide, anti-cancer peptide, and apoptosis inhibitory peptide, obtained by the screening method.

These and other advantages of the present invention will become apparent to those skilled in the art upon reading and understanding the following detailed description with reference to the accompanying figures.

### BRIEF DESCRIPTION OF THE DRAWINGS

The following drawings are illustrative of embodiments of the invention and are not meant to limit the scope of the invention as encompassed by the claims.

FIG. 1 shows an example of DNA library design.

FIG. 2 shows a screening scheme.

FIG. 3 shows the results of agarose gel electrophoresis of products after screening.

### DESCRIPTION OF THE PREFERRED EMBODIMENTS

(Definitions)

As used herein, the term “pathogenicity” refers to the capability of pathogens (including microorganisms, such as bacteria and the like) to infect organisms. As used herein, the term “animal pathogenic microorganism” refers to microorganisms capable of infecting animals. The term “plant pathogenic microorganism” refers to microorganisms capable of infecting plants. As used herein, the term “putrefactive microorganism” refers to microorganisms capable of causing putrefaction of food or industrial products.

As used herein, the term “library” refers to a population of sequences, preferably where the sequences are nucleic acid sequences. In one embodiment, the nucleic acid sequence is incorporated into a vector. As used herein, the term “nucleic acid sequence” may be used interchangeably with the term “base sequence”. The term “vector” refers to DNA capable of transporting homogenous or exogenous DNA to hosts and/or cell-free systems which carry out gene transcription and translation. Examples of nucleic acid sequences include, but are not limited to, nucleic acid sequences obtained by fragmentation of total DNA of a certain species or total DNA of a particular tissue or organ, and synthetic nucleic acid sequences. A particular library may be used to screen for DNA encoding a protein, amino acid or peptide having a desired property. As used herein, the term “screening” refers to a process that selects a compound having a desired property from newly synthesized compounds or compounds cloned from naturally occurring compounds. As used herein, the term “cloning” refers to a process that isolates a gene, a protein, an amino acid, or a peptide.

As used herein, the term “amino acid” includes a D-amino acid as well as an L-amino acid. D-amino acids naturally occur as components of peptide glycans present in the cell walls of bacteria and certain peptide antibiotics. As used herein, the term “peptide” refers to synthetic peptides as well as naturally-occurring peptides. Peptides are composed of L-amino acids or D-amino acids. Since it is not required to distinguish L-amino acid from D-amino acid, the cost of peptide synthesis may be largely removed when synthetic peptides are used for libraries.

Amino acids having a high probability of being present inside a protein in aqueous solution are referred to as “hydrophobic amino acids”, while amino acid having tendency to projecting toward the outside are referred to as “hydrophilic amino acids”. Specifically, “hydrophobic amino acids” include phenylalanine, tryptophan, isoleucine, leucine, proline, methionine, valine, alanine, and the like. “Hydrophilic amino acids” include lysine, glutamine, aspartate, glutamate, threonine, asparagine, arginine, serine, and the like. The hydrophobic region of the surface of a protein is considered to be linked to other proteins and the lipid portion of a membrane.

As used herein, the term “analog” with respect to peptides on Tables 1 and 2 refers to peptides that retain substantially the same biological function or activity. An analog includes a peptide where the C-terminal carboxyl group is blocked. A preferable example of the blockage of a C-terminal carboxyl group includes, but is not limited to, amidation. The term “biological function” refers to functions possessed by organisms in order to maintain viability, including, but not being limited to, for example, replication, transcription, and translation at the gene level, and catabolism, anabolism, and metabolism at the cell and individual levels, and the like. The term “biological activity” refers to activity which is measured by any biological assay. The term “analogs refers to biologically active derivatives of the reference molecule, or fragments of such derivatives, that retain desired activity in assays as described herein. In general, the term “analog” refers to compounds having a native polypeptide sequence and structure with one or more amino acid additions, substitutions (generally conservative in nature) and/or deletions, relative to the native molecule, so long as the modifications do not destroy desired activity. Preferably, the “analog” has at least the same activity as the native molecule. The activity of the analog may not be the same as the native peptide, i.e., may be higher or lower than the activity of the native peptide. Methods for making peptide analogs are known in the art and are described further below.

Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) hydrophobic—glycine, alanine, valine, leucine, isoleucine, proline, phenylalanine, methionine, tryptophan; and (4) neutral and hydrophilic—asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include 1, 2, 3, and 4 conservative or non-conservative amino acid substitutions, up to 5-15 conservative or non-conservative amino acid substitutions, or any integer between 5-15 of conservative or non-conservative amino acid substitutions, so long as the desired function of the molecule remains intact. One of skill in the art may readily determine regions of the molecule of interest that can tolerate change by reference to the Hopp/Woods method (Hopp et al., Proc. Natl. Acad. Sci. USA, 78:3824-3828 (1981)) and the Kyte-Doolittle method (Kyte et al., J. Mol. Biol., 157:105-132 (1982)), well known in the art.

In constructing variants of the polypeptide of interest, modifications are made such that variants continue to possess the desired activity. Biologically active variants of a polypeptide of interest will generally have at least 70%, preferably at least 80%, morepreferably about 90% to 95% or more, and most preferably about 98% or more amino acid sequence identity to the amino acid sequence of the reference polypeptide molecule, which serves as the basis for comparison. A biologically active variant of a native polypeptide of interest may differ from the native polypeptide by 1-15 amino acids, 1-10, such as 6-10, 5, 4, 3, 2, or even 1 amino acid residue. By “sequence identity” is intended the same amino acid residues are found within the variant polypeptide and the polypeptide molecule that serves as a reference when a specified, contiguous segment of the amino acid sequence of the variant is aligned and compared to the amino acid sequence of the reference molecule. The percentage sequence identity between two amino acid sequences is calculated by determining the number of positions at which the identical amino acid residue occurs in both sequences to yield the number of matched positions, dividing the number of matched positions by the total number of positions in the segment undergoing comparison to the reference molecule, and multiplying the result by 100 to yield the percentage of sequence identity.

For purposes of optimal alignment of the two sequences, the contiguous segment of the amino acid sequence of the variant may have additional amino acid residues or deleted amino acid residues with respect to the amino acid sequence of the reference molecule. The contiguous segment used for comparison to the reference amino acid sequence will comprise at least 20 contiguous amino acid residues, and may be 30, 40, 50, 100, or more residues. Corrections for changed sequence identity associated with inclusion of gaps in the variant's amino acid sequence can be made by assigning gap penalties. Methods of sequence alignment are well known in the art for both amino acid sequences and for the nucleotide sequences encoding amino acid sequences.

Thus, the determination of percent identity between any two sequences can be accomplished using a mathematical algorithm. One preferred, non-limiting example of a mathematical algorithm utilized for the comparison of sequences is the algorithm of Myers and Miller (1988) CABIOS 4:11-17. Such an algorithm is utilized in the ALIGN program (version 2.0), which is part of the GCG sequence alignment software package. APAM120 weight residue table, a gap length penalty of 12, and a gap penalty of 4 can be used with the ALIGN program when comparing amino acid sequences. Another preferred, nonlimiting example of a mathematical algorithm for use in comparing two sequences is the algorithm of Karlin and Altschul (1990) Proc. Natl. Acad. Sci. USA, 87:2264, modified as in Karlin and Altschul (1993) Proc. Natl. Acad. Sci. USA, 90:5873-5877. Such an algorithm is inserted into the NBLAST and XBLAST programs of Altschul et al. (1990) J. Mol. Biol., 215:403. BLAST nucleotide searches can be performed with the NBLAST program, score=100, wordlength=12, to obtain nucleotide sequences homologous to a nucleotide sequence encoding the peptide of interest. BLAST protein searches can be performed with the XBLAST program, score=50, word length=3, to obtain amino acid sequences homologous to the peptide of interest. To obtain gapped alignments for comparison purposes, Gapped BLAST can be utilized as described in Altschul et al. (1997) Nucleic Acids Res., 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Also see the ALIGN program (Dayhoff (1978) in Atlas of Protein Sequence and Structure 5: Suppl. 3 (National Biomedical Research Foundation, Washington, D.C.) and programs in the Wisconsin Sequence Analysis Package, Version 8 (available from Genetics Computer Group, Madison, Wis.), for example, the GAP program, where default parameters of the programs are utilized.

When considering the percentage of amino acid sequence identity, some amino acid residue positions may differ as a result of conservative amino acid substitutions, which do not affect properties of protein function. In these instances, percent sequence identity maybe adjusted upwards to account for the similarity in conservatively substituted amino acids. Such adjustments are well known in the art. See, for example, Myers and Miller (1988), Computer Applic. Biol. Sci., 4:11-17.

The precise chemical structure of a peptide depends on a number of factors. As ionizable amino and carboxyl groups are present in the molecule, a particular peptide may be obtained as an acidic or basic salt, or in neutral form. All such preparations that retain their biological activity when placed in suitable environmental conditions are included in the definition of peptides as used herein. Further, the primary amino acid sequence of the peptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of peptide analog used herein so long as the activity of the peptide is not destroyed. It is expected that such modifications may quantitatively or qualitatively affect the activity, either by enhancing or diminishing the activity of the peptide, in the various assays. Further, individual amino acid residues in the chain may be modified by oxidation, reduction, or other derivatization, and the peptide may be cleaved to obtain fragments that retain activity. Such alterations that do not destroy activity do not remove the peptide sequence from the definition of peptide analog of interest as used herein.

As used herein, the term “flexible” refers to a condition of a plurality of peptide or protein-derived fragments such that they move freely without mutual interference. Preferably, the plurality of peptide or protein-derived fragments are linked with a linker sequence (also referred to as a linker or linker peptide). As used herein, the term “linker sequence” refers to an amino acid sequence having 3, 4, 5, 6, 7, 8, 9, 10, or 10 or more contiguous amino acid residues. In one embodiment, the linker sequence may include SEQ ID NO: 4. In a preferred embodiment, the linker sequence may have two or more repeated SEQ ID NO: 4.

In one embodiment, the peptide of the present invention is linked with a linker sequence for allowing flexible movement and a tag sequence for prescreening. The term “prescreening” refers to screening prior to screening (also referred to as main screening) for a substance having a desired property. In one aspect, prescreening may be carried out based on a property different from a feature of the peptide of the present invention. The term “tag sequence” refers to amino acid sequences that are linked with antibodies or affinity resins. The tag sequence may be derived from the same or different species than the peptide of the present invention. In a preferred embodiment, the tag sequence is a heterologous epitope. Examples of heterologous epitopes include, but are not limited to, FLAG, myc, HA, SV40 T antigen, glutathione S-transferase, 6 histidine, and maltose binding protein.

As used herein, the terms biological membranes and “cell membrane” are used interchangeably to refer to intact cell membrane structure. The term “membrane model” refers to a membrane structure that is constructed by imitating a biological membrane. The term “imitate” with respect to the cell membrane structure of organisms refers to possession of all or a part of the property and feature of the intact cell membrane structure of an organism. Preferably, the membrane model imitates the cell membrane structure of different species by changing the type and/or proportion of lipids contained in the membrane. In one aspect, a membrane model used in the present invention may be a liposome having an artificial lipid bilayer. An example of a model of the cell membrane structure of microorganisms includes, but is not limited to, a membrane containing an acid phospholipid, such as phosphatidyl glycerol, phosphatidyl serine, or the like, without cholesterol. An example of a model of the cell membrane structure of a healthy animal includes, but is not limited to, a membrane containing cholesterol but not an acid phospholipid.

The liposome preparation is any of the liposome constructs which are a large unilamellar vesicle (LUV), a multilammelar vesicle (MLV), and a small unilamellar vesicle (SUV). The LUV has a particle diameter ranging from about 200 to about 1000 nm. The MLV has a particle diameter ranging from about 400 to about 3500 nm. The SUV has a particle diameter ranging from about 20 to about 100 nm.

Liposomes are prepared by a number of common methods, including, but not limited to, reverse-phase evaporation (Szoka, F., et al: Biochim. Biophys. Acta, Vol. 601, 559 (1980)); ether injection (Deamer, D. W., Ann. N. Y. Acad. Sci., Vol.308,250(1978)); and a surfactant method (Brunner, J., et al, Biochim. Biophys. Acta, Vol. 455, 322 (1976)).

As lipids for forming liposomes, phospholipids, cholesterols, nitrogen-containing lipid (e.g., glycolipids), and the like are used. Generally and preferably, phospholipids are used. Examples of phospholipids include, but are not limited to, naturally-occurring phospholipids (e.g., phosphatidyl choline, phosphatidyl serine, phosphatidyl glycerol, phosphatidyl inositol, phosphatidyl ethanolamine, phosphatidate, cardiolipin, sphingomyelin, egg-yolk lecithin, soya lecithin, and lysolecithin, etc.), or those supplemented with hydrogen using a common method; synthetic phospholipids (e.g., dicetyl phosphate, distearoyl phosphatidyl choline, dipalmitoylphosphatidyl choline, dipalmitoylphosphatidyl ethanolamine, dipalmitoylphosphatidyl serine, eleostearoyl phosphatidyl choline, eleostearoyl phosphatidyl ethanolamine, eleostearoyl phosphatidyl serine, etc.).

The lipids including these phospholipids may be used alone or in combination. In forming liposomes, a liposome forming additive (e.g., cholesterols, stearyl amine, α-tocopherol, etc.) may be used in combination with major phospholipids.

Liposomes are prepared by, for example, dissolving the above-described liposome forming substance, cholesterol and the like in an organic solvent (e.g., tetrahydrofuran, chloroform, ethanol, etc.), and placing the solution in an appropriate container under reduced pressure to evaporate the solvent, so that a film of the liposome forming substance is formed on the inner wall of the container. A buffer solution is added to the resultant film, followed by stirring. If desired, the above-described membrane fusion promoting substance is added before isolating liposomes. The resultant liposome may be suspended in an appropriate solvent or may be lyophilized and, if required, dispensed in an appropriate solvent. The membrane fusion promoting substance may be added after isolation of a liposome and before use.

As used herein, the term “cell free extract solution” refers to soluble fractions obtained by homogenizing cells followed by centrifugation of cell debris solution. The “cell free extract solution” contains a ribosome, an aminoacyl tRNA synthetase (ARS), a polypeptide chain initiation factor (IF), a polypeptide chain elongation factor (EF), and a polypeptide chain termination factor (RF), which are required for translation of mRNA.

As used herein, the term “treating” or “treatment” refers to the reduction or alleviation of one or more symptoms possessed by a predetermined cell or individual, the prevention of one or more symptoms from worsening or proceeding, the promotion of recovery or the amelioration of prognosis, and/or the prevention of disease, and the retardation or attenuation of the progression of existing diseases. For a predetermined individual, the amelioration, deterioration, regression, or progression of symptoms may be determined by objective or subjective measurement. The efficacy of treatment may be measured by an improvement in morbidity or mortality (e.g., the extension of the survival curve of a selected population). Prevention methods (e.g., prevention or reduction of recurrence) are also regarded as treatment. Treatment may include a combination with other existing treatments (e.g., one or more other drugs and one or more other medical procedures).

As used herein, the term “abnormal cell” refers to cells, such as apoptotic cells, cancer cells, and the like, which have an abnormality compared to healthy animal cells. Preferably, the abnormal cell refers to animal cells in which acid phospholipids are exposed to the outside of the cells. The term “healthy cell” refers to normal or healthy cells, and preferably refers to animal cells in which acid phospholipids are not exposed to the outside of the cells.

As used herein, “at least two” refers to any integer greater than or equal to two. Preferably, “at least two” refers to 2, 3, 4, 5, 6, 7 or 8.

As used herein, the term “acting” with respect to biological membranes refers to the targeting of biological membranes, including the attaching and/or binding of biological membranes, and the penetration of biological membranes. In a preferred embodiment, the peptide of the present invention may suppress or inhibit cell functions by acting on a specific cell membrane. In more preferred embodiment, the cell maybe any of microorganism cells, cancer cells or apoptotic cells. “Microorganism” herein includes pathogenic microorganisms, putrefactive microorganisms, and the like. In another embodiment, the peptide of the present invention may promote cell functions by acting on the membrane of a specific cell. In still another embodiment, the peptide of the present invention may act on biological membranes without affecting the function of a specific cell. It will be easily understood by those skilled in the art that the peptide of the present invention is useful for delivery of a desired substance (e.g., a drug, a gene, etc.) to a specific cell.

(A. Method for Screening for a Peptide Capable of Acting on a Biological Membrane of Interest)

The present inventors used two membrane perturbation peptides (mast21 (SEQ ID NO: 5) and mastoparanX (SEQ ID NO: 10)) as a reference so as to construct a method for screening for a peptide capable of acting on a biological membrane of interest. Mast21 specifically acts on the membrane of microorganisms but not on the membrane of animal cells. MastoparanX acts both on the membrane of microorganisms and the membrane of animal cells. Specifically, an attempt has been made to establish the following method for screening a peptide capable of acting on a cell membrane structure, where the two peptides are subject peptides: when a microorganism membrane model is used as an immobilized membrane model, both mast21 and mastoparanX are screened for: when an animal cell membrane model is used, only mastoparanX is screened for; and the full-length amino acid sequence of a peptide is obtained without a deletion. We considered that such a method would be appropriate for screening random peptide libraries for a peptide capable of acting on a cell membrane structure. However, both of these peptides have potent antibacterial activity, and therefore, cannot be screened for or, if possible, is screened with some deletions, in a method including a step utilizing the function of organisms, such as phage display, bacteria surface display, yeast surface display, or the like. We found that if the full screening process is carried out in cell-free systems (in vitro), i.e., without utilizing the function of living cells, such as microorganisms, phages, or the like, it is possible to fully remove the influence of a peptide on organisms during the screening process even if the peptide is lethal to the organisms or there is the possibility that the peptide has an influence on the growth of the organisms. In other words, with such an in vitro screening method, it is possible to significantly reduce the possibility that an amino acid sequence expected to act on organisms is partially deleted or removed.

The present inventors also found that by designing and constructing libraries for transcription and translation in cell-free systems where a first cassette and a second cassette are operatively linked with a third cassette, the amino acid sequence of a peptide can be associated with genetic information, so that random peptide libraries can be efficiently expressed. The library of the present invention in which a random peptide library can be efficiently expressed is a library comprising a plurality of nucleic acid sequences, where each of the nucleic acid sequences comprises the following three cassettes:

(1) a first cassette comprising a base sequence encoding a first peptide;

(2) a second cassette comprising a base sequence encoding a second peptide in the same reading frame as the base sequence encoding the first peptide, where the second peptide comprises a site which allows the first peptide to move flexibly; and

(3) a third cassette comprising a base sequence essential for the transcription and translation of the first and second cassettes, and operatively linked with the first and second cassettes,

where the library comprises at least two nucleic acid sequences having different first cassettes. Specifically, a base sequence essentially required for transcription and translation is incorporated into the upstream cassette, a DNA library, which has been designed in accordance with a peptide of interest, is inserted into the middle cassette, and a site, which allows the expressed peptide to move flexibly, is inserted into the downstream cassette. Preferably, the DNA library consists of DNAs lacking termination codons. The second and third cassettes can be commonly used in all libraries. Therefore, the first, second and third cassettes linked together allow simultaneous transcription and translation in cell-free systems. As a result, a peptide as a product of translation and mRNA as a product of transcription forms a complex, so that it is possible to create a peptide library in which the amino acid sequence of a peptide of interest can be easily associated with genetic information.

We designed and constructed DNA libraries comprising the base sequences of genes encoding the above-described two peptides (mast21 and mastoparanX). Transcription and translation were carried out in cell-free systems to form peptide-ribosome-mRNA (PRM) complexes. Thereafter, by increasing the magnesium concentration, the PRM complexes were stabilized. A peptide capable of specifically acting on an immobilized membrane model was efficiently screened for and concentrated. Finally, a full-length peptide was obtained. Specifically, a base sequence encoding the amino acid sequence of mast21 or mastoparanX was inserted into a cassette. PRM complexes were formed in cell-free systems. Screening was carried out using an immobilized membrane model. The genetic information of a peptide having a binding capability was confirmed.

The present inventors developed a technique for efficiently concentrating a peptide capable of acting on the membrane of microorganisms. In this technique, the selection pressure for screening was considered such that liposomes having an artificial lipid bilayer, which imitates the cell membrane structure of various organisms in accordance with the purposes, are immobilized onto a solid phase and the resultant structure functions as an immobilized membrane model. A peptide capable of binding to the immobilized membrane model was efficiently recovered as a peptide-ribosome-mRNA complex (PRM complex) from cell-free systems.

Thereafter, the PRM complexes specifically binding to the immobilized membrane model were recovered together with magnetic beads. Only mRNA was purified from the PRM complex. The mRNA was reverse-transcribed into DNA by RT-PCR. Therefore, the above-described cycle could be repeated. It is believed that by repeating the screening cycle several times or more, a peptide capable of specifically acting on the membrane of microorganisms can be concentrated. After the last cycle, by determining the base sequence of the resultant DNA, the amino acid sequence of the peptide capable of specifically binding to the immobilized membrane model was determined.

In one aspect, the present invention provides a method for screening a nucleic acid encoding a peptide capable of acting on a biological membrane. The method comprises the steps of:

designing and constructing a DNA library;

forming a peptide-ribosome-mRNA complex by transcription and translation of the library in a cell-free system; and

selecting the complex specifically binding to a membrane model.

(B. Peptide Obtained by the Method of the Present Invention)

With the above-described method, it is possible to screen for a novel peptide capable of specifically acting on a biological membrane. In one aspect, the biological membrane may be the membrane of a microorganism. Preferably, the microorganism may be a pathogenic microorganism or a putrefactive microorganism. In another aspect, the biological membrane may be of a eukaryotic organism. Preferably, the eukaryotic organism may be a mammalian animal cell. More preferably, the mammalian animal cell may be a cancer cell or an apoptotic cell. In another aspect, the biological membrane may be a membrane of a microorganism cell or a eukaryotic cell.

The biological membrane of a cell (e.g., an apoptotic cell, a cancer cell, etc.) having an abnormality in the cell membrane compared to healthy animal cells is also specifically recognized by the peptide of the present invention since acid phospholipids are exposed toward the outside of the cell. Therefore, it will be easily understood by those skilled in the art that the screening method of the present invention may be used to obtain not only a novel peptide capable of specifically acting on the membrane of microorganisms, but also a peptide capable of recognizing and acting on cancer cells and apoptotic cells, preferably, a peptide capable of killing cancer cells and a peptide capable of suppressing apoptosis (hereafter referred to as a “apoptosis inhibitory peptide”).

The present invention also provides a peptide capable of acting on a biological membrane more strongly than mast21. Preferably, the peptide significantly acts on a biological membrane more strongly than mast21 by a factor of at least 2, preferably by a factor of at least 3, 4, or 5, more preferably by a factor of at least 6, 7, 8, 9, or 10. In a preferred embodiment, the peptide is mast21R (SEQ ID NO: 107).

The peptide of the present invention may be directed to cancer and/or apoptosis. Therefore, the peptide of the present invention is useful in the treatment of a number of diseases relating to cancer and/or apoptosis. Examples of diseases which may be treated by the peptide of the present invention, include cancers (including, but not limited to, follicular lymphomas, carcinomas with p53 mutations, and hormone-dependent tumors (colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, kidney cancer, uterine cancer, bladder cancer, Kaposi's sarcoma and ovarian cancer)); autoimmune disorders (such as, multiple sclerosis, Sjogren's syndrome, Hashimoto's thyroiditis, biliary cirrhosis, Behcet's disease, Crohn's disease, polymyositis, systemic erythematosus and immune related glomerulonephritis and rheumatoid arthritis); and viral infections (such as herpes viruses, pox viruses and adenoviruses), inflammation, graft versus host disease, acute graft rejection, and chronic graft rejection. In a preferred embodiment, the peptide of the present invention is used to inhibit growth, progression, and/or metasis of cancers, in particular those listed above.

Additional diseases or conditions associated with cell survival that may be treated or detected by the peptide of the present invention, include, but are not limited to, growth, and/or metastases of the following malignant tumors and related disorders: leukemia (including acute leukemias (e.g., acute lymphocytic leukemia, acute myelocytic leukemia (including myeloblastic, promyelocytic, myelomonocytic, monocytic, and erythroleukemia)), and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors (including, but not limited to, sarcomas and carcinomas (such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synoviosarcoma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma)).

Diseases associated with progression of apoptosis that may be treated or detected by the peptide of the present invention include: AIDS; neurodegenerative disorders (such as Alzheimer's disease, Parkinson's disease, amyotrophic lateral sclerosis, retinitis pigmentosa, cerebellar degeneration and brain tumor or prior associated disease); autoimmune disorders (such as, multiple sclerosis, Sjogren's syndrome, Hashimoto's thyroiditis, biliary cirrhosis, Behcet's disease, Crohn's disease, polymyositis, systemic erythematosus and immune-related glomerulonephritis and rheumatoid arthritis), myelodysplastic syndromes (such as aplastic anemia), graft versus host disease, ischemic injury (such as that caused by myocardial infarction, stroke and reperfusion injury), liver injury (e.g., hepatitis related liver injury, ischemia/reperfusion injury, cholestosis (bile duct injury) and liver cancer); toxin-induced liver disease (such as that caused by alcohol), septic shock, cachexia and anorexia.

In a preferred embodiment, a peptide having a sequence obtained by the method of the present invention is chemically synthesized, and the ability of the peptide to acting on a cell membrane structure can be investigated using, for example, a fluorescent substance (calcein) containing model membrane (liposome) (see, e.g., Japanese Patent No. 2967925). In another embodiment, a peptide having a sequence obtained by the method of the present invention may be evaluated in accordance with U.S. National Committee for Clinical Laboratory Standard (NCCL Documents M7-A3). In another embodiment, a peptide having a sequence obtained by the method of the present invention may be evaluated with respect to the reactivity to normal animal cells by investigating the hemolytic property with respect to erythrocytes obtained by centrifuging fresh blood collected from animals. In still another embodiment, a peptide having a sequence obtained by the method of the present invention may be evaluated with respect to the specificity to cancer cells and the effect of killing cancer cells by causing the peptide to act on various tumor cell lines. In even still another embodiment, a peptide having a sequence obtained by the method of the present invention may be evaluated with respect to the ability to act on apoptotic cells by causing the peptide to act on cells and/or cell lines in which apoptosis is induced. In another embodiment, a peptide having a sequence obtained by the method of the present invention may be evaluated with respect to the effect of suppressing and/or inhibiting apoptosis by causing the peptide to act on various apoptotic cells.

It will be apparent for those skilled in the art that the above-described method can be easily modified to adapt to screen for a peptide specific to various cell membranes. It is possible for those skilled in the art to prepare a membrane model for various cells (e.g., mammalian animal cells, microorganism cells, etc.) by changing the type and/or proportion of a lipid contained in the membrane model. For example, the ratio of phospholipids in a membrane model which imitates a cell membrane structure of an organism is well known. In the case of the membrane of a mammalian animal cell, an exemplary membrane model has a ratio, PC:Ch1=10:1 or PC:Sph:Ch1=3:3:2. In the case of the membrane of a microorganism, an exemplary membrane model has a ratio, PC: PG=7:3, PE:PG=7:3, PC:PS=1:1, PS (or PE) :PG:CL=7:2:1, or PC:PG:CL=3:3: 2(where PS represents phosphatidyl serine, PC represents phosphatidyl choline, PG represents phosphatidyl glycerol, PE represents phosphatidyl ethanolamine, Ch1 represents cholesterol, Sph represents sphingomyelin, and CL represents cardiolipin). The present invention is not limited to the above-described examples. Preferably, the above-described membrane model may contain PA (phosphatidate), LPS (lipopolysaccharide), or lipid A. A liposome containing PG is more stable than a liposome containing PS. Therefore, those skilled in the art may use PG and PS interchangeably when producing membrane models. With the above-described well-known technique, it is possible to easily obtain a peptide capable of specifically acting on a desired cell membrane. In addition, the toxicity of obtained peptides with respect to any organism may be easily evaluated by those skilled in the art by carrying out a toxicity test.

Peptides screened for by the above-described method may be used in various applications. Preferably, when the peptide of the present invention is applied to infectious diseases, the peptide may function as a host defense peptide which selectively exhibits toxicity to a target cell without damaging host cells or normal cells. In one embodiment, the target cell may be a pathogenic microorganism. In another embodiment, the target cell may be a cancer cell. In another embodiment, the target cell maybe an “apoptotic cell” which is undergoing apoptosis. Examples of applications preferable for the peptide include, but are not limited to, pharmaceutical compositions for killing microorganisms, pharmaceutical compositions for treating infectious diseases caused by microorganisms, antibiotics, pharmaceutical delivery substances for delivering a drug to a site infected with microorganisms, pharmaceutical compositions for treating cancers, pharmaceutical delivery substances for delivering a drug to cancer lesion sites, pharmaceutical compositions for suppressing apoptosis, and pharmaceutical substances delivered to apoptosis progression sites.

(C. Pharmaceutical Composition)

The present invention discloses a method for treating a subject by administering an effective amount of the pharmaceutical composition of the present invention. In a preferred embodiment, the pharmaceutical composition is substantially pure. The subject is preferably an animal. The animal is preferably a mammalian animal, most preferably a human.

The method and route of administration of the pharmaceutical composition may be selected from the following.

As used herein, the term “pharmaceutical delivery substance” refers to compositions including pharmaceutical composition delivery systems. A number of types of pharmaceutical composition delivery systems are well known in the art and may be used to administer the pharmaceutical composition of the present invention. Examples of the delivery systems include, but are not limited to, (i) encapsulation in liposomes, microparticles, and microcapsules; (ii) recombinant cells capable of expressing the pharmaceutical composition; (iii) receptor-mediated endocytosis (see, e.g., Wu and Wu, 1987, J. Biol. Chem., 262:4429-4432); (iv) construction of a pharmaceutical composition nucleic acid as part of a retroviral or other vector, and the like. The peptide of the present invention may be used as a delivery system for delivery of a desired pharmaceutical to a target disease site (e.g., microorganisms, cancer cells, or apoptotic cells). Methods of administration/introduction include, but are not limited to, intradermal, intramuscular, intraperitoneal, intravenous, subcutaneous, intranasal, epidural, and oral routes. The pharmaceutical composition of the present invention may be administered by any convenient route, for example, by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the pharmaceutical composition of the present invention into the central nervous system by any suitable route, including intraventricular, intrathecal, and subdural injection. Intraventricular injection may be facilitated by an intraventricular catheter attached to a reservoir. Pulmonary administration may also be employed by use of an inhaler or nebulizer, and formulation with an aerosolizing agent.

In a specific embodiment of the present invention, it may also be desirable to administer the pharmaceutical composition of the present invention locally to the area in need of treatment; this may be achieved by, for example, and not by way of limitation, local infusion during surgery, topical application (e.g., in conjunction with a wound dressing after surgery), by injection, by means of a catheter, by means of a suppository, or by means of an implant, said implant being of a porous, non-porous or gelatinous material, including membranes or fibers).

In another specific embodiment of the present invention, the pharmaceutical composition can be delivered in a vesicle, in particular a liposome (see Langer, 1990, Science, 249: 1527-1533). In yet another embodiment, the pharmaceutical composition can be delivered in a controlled release system, including, but not limited to, a pump (see Sefton, 1987, CRC Crit. Ref. Biomed. Eng., 14: 201) and a polymer substance (e.g., Smolen and Ball, 1983, Controlled Drug Bioavailability, Drug Product Design and Performance (Wiley, New York, N.Y.)). Additionally, the controlled release system can be placed in proximity of the therapeutic target (e.g., the brain), thus requiring only a fraction of the systemic dose. See, Goodson, 1984, Medical Applications of Controlled Release, (Wiley, New York, N.Y.).

The present invention also discloses a pharmaceutical composition. The composition comprises a therapeutically effective amount of pharmaceutical composition within a pharmaceutical acceptable carrier. In a specific embodiment, the term “pharmaceutical acceptable” as used herein means approved by a government or listed in the Pharmacopeia for use in animals, and more particularly in humans. As used herein, the term “carrier” refers to a diluent, adjuvant, excipient, or vehicle with which the therapeutic agent is administered together. Such pharmaceutical carriers include, but are not limited to, sterile liquids (e.g., water, saline, etc.) and oils (e.g., those of petroleum, animal, vegetable or synthetic origin, such as peanut oil, soybean oil, mineral oil, sesame oil and the like). Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Martin, 1965, Remington's Pharmaceutical Sciences. Such compositions will contain a therapeutically effective amount of the pharmaceutical composition, preferably in purified form, and most preferably in a substantially purified form, together with a suitable amount of carrier so as to provide the form for proper administration to the patient. The formulation should suit the mode of administration.

In a preferred embodiment of the present invention, the composition is formulated in accordance with routine procedures as a pharmaceutical composition adapted for compositions for intravenous administration are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampoule indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical composition of the present invention includes a pharmaceutically acceptable salt, including salts derived from hydrochloric, phosphoric, acetic acids, etc., and salts formed by using free carboxyl radicals (for example, salts derived from sodium, potassium, calcium, ferric hydroxide, isopropylamine, triethylamine, 2-ethylaminoethanol, histidine, procaine, etc.).

The amount of the pharmaceutical composition of the present invention which will be effective in the treatment of a particular disorder or condition will depend on the nature of the disorder or condition, and may be quantitatively determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20 to 500 μg of active compound per kilogram (Kg) body weight. Suitable dosage ranges for intranasal administration are generally about 0.01 pg/kg body weight to about 1 mg/kg body weight. Effective doses maybe extrapolated from dose-response curves derived from in vitro or animal model test systems. Suppositories generally contain active ingredients in the range of 0.5% to 10% by weight; oral formulations preferably contain 10% to 95% active ingredients.

(D. Kit)

The present invention also provides a pharmaceutical pack or kit, comprising one or more containers filled with one or more of the ingredients of the pharmaceutical compositions of the present invention. Optionally associated with such container(s) may be a notice in the form prescribed by a governmental agency regulating the manufacture, use or sale of pharmaceuticals or biological products, which notice reflects approval by the agency of manufacture, use or sale for human administration.

In another embodiment, the present invention may be assembled into a kit for use in reverse-transcription or amplification of a nucleic acid molecule or a kit for use in sequencing of a nucleic acid molecule. Kits according to this aspect of the present invention may comprise a carrier, such as a box, carton, tube or the like, having in close confinement therein one or more containers, such as vials, tubes, ampoules, bottles, and the like. A first container contains a peptide having reverse-transcriptase activity. When one or more peptides having reverse-transcription activity are employed, the mixture of two or more peptides may be stored in a single or separate containers. The kit of the present invention may contain in the same or separate containers one or more DNA polymerase, appropriate buffer solution, one or more nucleotides, and/or one or more primers.

In a particular aspect of the present invention, the reverse-transcription and amplification kits may comprise one or more ingredients (as a mixture or separately). These ingredients include libraries including a nucleic acid sequence encoding the peptide of the present invention, one or more nucleotides required for synthesis of nucleic acid molecules, an oligo (dT) for reverse-transcription, and/or one or more primers. The reverse-transcription and amplification kits may further comprise one or more DNA polymerases. The sequencing kit of the present invention may comprise a library including a nucleic acid sequence encoding the peptide of the present invention, and optionally one or more DNA polymerase, and one or more reaction terminating agents (e.g., a dideoxynucleoside triphosphate molecule) required for sequencing of nucleic acid molecules, one or more nucleotides, and/or one or more primers. Preferable peptides having reverse-transcriptase activity, DNA polymerases, nucleotides, primers, and other ingredients appropriate for use in the reverse-transcription, amplification, and sequencing kits of the present invention are illustrated above. The kit according to this aspect of the present invention may comprise additional reagents and compounds required for standard nucleic acid reverse-transcription, amplification or sequencing methods. The peptide of the present invention having reverse-transcriptase activity, a DNA polymerase, a nucleotide, a primer, and an additional reagent, ingredient or compound may be contained in one or more container. A mixture of at least two of the above-described ingredients may be contained in such a container or in separate containers in the kit of the present invention.

(E. Antibody Production)

The present invention provides antibodies against a peptide obtained by the screening method of the present invention. Immunogens for raising antibodies are prepared by mixing the peptide of the present invention with adjuvants. Alternatively, peptides are made as fusion proteins to larger immunogenic proteins. Peptides are also covalently linked to other larger immunogenic proteins, such as keyhole limpet hemocyanin. Immunogens are typically administered intradermally, subcutaneously, or intramuscularly. Immunogens are administered to experimental animals such as rabbits, sheep, and mice, to generate antibodies. Optionally, the animal spleen cells are isolated and fused with myeloma cells to form hybridomas which secrete monoclonal antibodies.

Preparations of polyclonal and monoclonal antibodies specific for selected peptides are made using standard methods known in the art. The antibodies specifically bind to epitopes present in the peptides disclosed in the sequence listing. Typically, at least about 6, 8, 10, or 12 contiguous amino acids are required to form an epitope. A short sequence of a peptide may then be unsuitable for use as an epitope to raise antibodies for identifying the corresponding novel protein, because of the potential for cross-reactivity with a known protein. However, the antibodies may be useful for other purposes, particularly if they identify common structural features of a known protein and a novel peptide of the present invention.

Antibodies that specifically bind to the peptide should provide a detection signal at least about 5-, 10-, or 20-fold higher than a detection signal provided with other proteins when used in Western blots or other immunochemical assays. Preferably, antibodies that specifically bind to the peptide of the present invention do not bind to other proteins at a detectable level in immunochemical assays and can immunoprecipitate the specific peptide from solution.

To test for the presence of serum antibodies to the peptide of the present invention in a human population, human antibodies may be purified by methods well known in the art. Preferably, the antibodies are affinity purified by passing antiserum over a column to which the corresponding selected peptide or fusion protein is bound. The bound antibodies can then be eluted from the column, for example, using a buffer with a high salt concentration.

Hereinafter, the method of the present invention for screening for a peptide capable of acting on a biological membrane, and a novel peptide obtained by the method of the present invention, will be described in greater detail.

### DETAILED DESCRIPTION OF THE INVENTION

Libraries including cassettes comprising base sequences encoding a plurality of peptides and lacking a termination codon are designed and constructed. Preferably, the base sequence encoding a peptide lacks a termination codon. If a termination codon is present, a peptide-ribosome-mRNA complex (PRM complex) as a result of transcription and translation (described below) cannot be maintained in the interconnected state that the peptide and the mRNA are coupled via the ribosome, which is not preferable. However, as described below, DNA having an interrupting termination codon or a frame shift is removed by prescreening before main screening. Therefore, it will be understood by those skilled in the art that DNA libraries employed are not necessarily limited to those lacking a termination codon and DNA libraries are prepared depending on a peptide of interest.

In a preferred embodiment, a library used in the present invention may be a DNA library of base sequences encoding a peptide consisting of 21 random amino acids. In a preferred embodiment, the base sequence may be a sequence (XXB)₂₀XAG lacking a termination codon, where X is A, T, G or C, and B is T or G.

The prepared DNA library comprises a first cassette, a second cassette, and a third cassette. These cassettes are operatively linked together so that the amino acid sequence of a peptide can be associated with genetic information, and the random peptide library can be efficiently expressed. Specifically, in the library of the present invention, the three cassettes, i.e., the first cassette, the second cassette, and the third cassette, are operatively linked together, where the upstream third cassette comprises a base sequence essentially required for transcription and translation; the middle first cassette comprises a nucleic acid sequence of a DNA library designed based on a peptide of interest, preferably a DNA library consisting of DNAs lacking a termination codon: and the downstream second cassette comprises a site that allows the expressed peptide to move freely. The main portion of the DNA library is composed of a plurality of the first cassettes, which corresponds to a library of base sequences to be subjected to screening. Various nucleic acid sequences designed based on a peptide of interest, preferably lacking a termination codon, re inserted into the middle first cassette, so that a plurality of first cassettes are constructed and used as a library and therefore various base sequences can be subjected to screening.

Even if the three cassettes are ligated together in an order of sequence other than the sequence of the third cassette, the first cassette, and the second cassette, the present invention can be carried out.

The third cassette comprises a base sequence essentially required for transcription and translation of the first and second cassettes. A DNA sequence in a vector is operatively linked to an appropriate control sequence of expression (promoter), and instructs the synthesis of mRNA. Typical examples of such a promoter include, but are not limited to, LTRs or the SV40 promoter, E. coli lac or trp, the λ phage P_{L} promoter, and other promoters known to control gene expression in prokaryotic or eukaryotic cells, or viruses. The vector may further comprise a ribosome binding site for initiating translation. The vector may further comprise a sequence appropriate for amplification of expression. Where necessary, a5′ stem-loop (SEQ ID NO: 3) may be inserted into the third cassette in order to stabilize mRNA transcribed from the first and second cassettes. The vector may further comprise a restriction enzyme site (e.g., NdeI site) on the 3′ side in order to facilitate ligation to the first cassette.

The first cassette may contain restriction enzyme sites (e.g., NdeI site and XbaI site) at a 5′ site and a 3′ site in order to facilitate the ligation to the third cassette and the second cassette, respectively. By changing the base sequence to be inserted into the first cassette, various peptides having a different length, randomness, expected structure or property, and the like, can be subjected to screening. As described above, the base sequence to be inserted into the first cassette preferably lacks a termination codon in order to maintain a peptide-ribosome-mRNA complex (PRM complex) obtained as a result of transcription and translation in the interconnected state that the peptide and the mRNA are coupled via the ribosome.

The second cassette may comprise a 3′ stem-loop for enhancing the stability of mRNA and a linker sequence (a sequence having a four contiguous SEQ ID NO: 4×2) for allowing the flexible movement of a translated peptide. When prescreening described below is carried out, a tag, such as a heterologous epitope for which homologous antibodies or affinity resins can be employed, (e.g., FLAG, myc, the SV40 T antigen, glutathione S-transferase, 6 histidine, maltose binding protein), may be inserted into the second cassette. The length of the linker sequence may be optionally extended or shortened where a unit length is a four contiguous SEQ ID NO: 4×2. A restriction enzyme site (e.g. XbaI site) may be introduced to the 5′ side of the second cassette in order to facilitate ligation to the linker second cassette encoding an amino acid sequence which can be repeated arbitrary times.

In one aspect, the common third cassette and second cassette are used, while only the first cassette may be prepared based on a peptide of interest. The first cassette is designed and prepared by chemical synthesis so that the base sequence of a gene encoding a peptide of interest capable of acting a cell membrane structure, which is to be subjected to screening, lacks a termination codon. For example, such a peptide is mast21 which is capable of specifically acting on the membrane of microorganisms. This DNA is amplified by PCR and is ligated with the second cassette, followed by further amplification by PCR. The DNA is cleaved with a restriction enzyme and is ligated with the upstream cassette, followed by PCR amplification. The resultant DNA is a template DNA which will be subjected to a transcription and translation system.

Thereafter, in a cell-free system, screening is carried out, which comprises the steps of: forming peptide-ribosome-mRNA complexes by transcription and translation of a library: and selecting a complex capable of specifically binding to a membrane model from the complexes. Preferably, the library contains DNAs lacking a termination codon. Preferably, the membrane model is an artificial lipid bilayer which imitates a cell membrane structure of organisms. Preferably, screening is carried out in vitro in a cell-free system, i.e., without using living cells, such as microorganisms, phages, or the like. If instead of cell-free systems, a technique for synthesizing proteins in vivo, such as phage display, bacteria surface display, yeast surface display, and the like, which employs living cells, is employed, the technique includes the step of employing organisms, such as microorganisms, phages, or the like, and therefore, it is not possible to select the amino acid sequence of a peptide having a potential to be lethal to organisms or affect the growth of organisms.

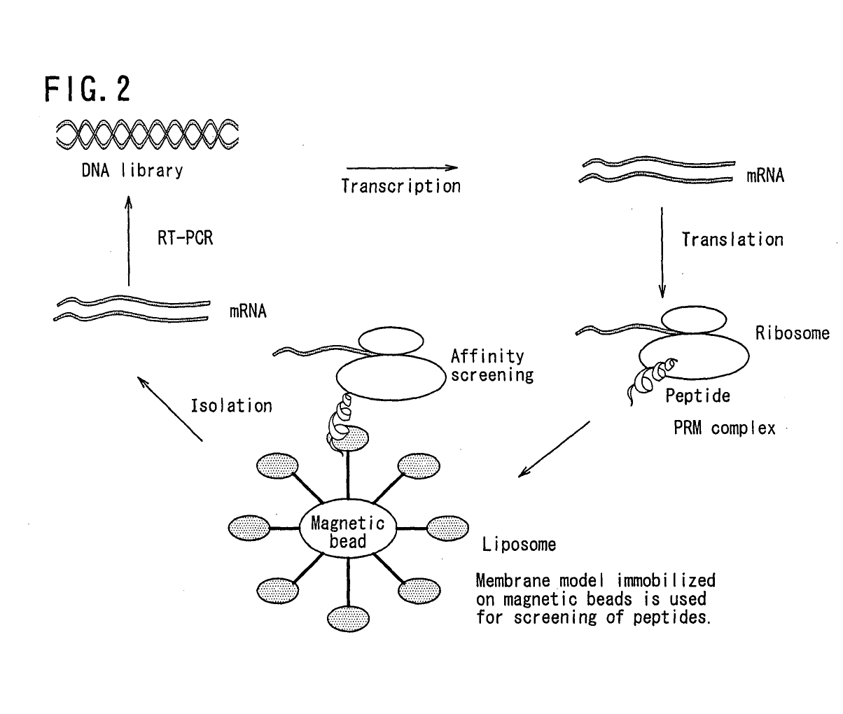

Screening comprising the steps of: forming peptide-ribosome-mRNA complexes (PRM complexes) by transcription and translation of a library of the present invention; selecting a PRM complex capable of specifically binding to an immobilized membrane model from the complexes; and reverse-transcribing mRNA in the selected PRM complex into DNA, can be carried out in accordance with the scheme shown in FIG. 2.

The step of forming peptide-ribosome-mRNA complexes (PRM complexes) by transcription and translation of a library of the present invention, is carried out under the condition that mRNA as a product of transcription and a peptide as a product of translation can form a stable complex via a ribosome. Specifically, the transcription and translation of a DNA library lacking a termination codon are carried out in vitro to form PRM complexes using a cell free extract solution of E. coli, a plant, or an animal. For example, a cell free extract solution of E. coli is obtained as a soluble fraction (referred to as S30 extract) by homogenizing the cell and centrifuging the cell debris solution at 30,000×g. The S30 extract contains a ribosome, aminoacyl tRNA synthetase (ARS), a polypeptide chain initiation factor (IF), a polypeptide chain elongation factor (EF), and a polypeptide chain termination factor (RF), which are required for translation of mRNA. The cell free extract solution is not limited to one derived from E. coli, and may be derived from eukaryotic organisms (e.g., rabbit reticulocytes, wheat embryos, etc.). It will be readily understood by those skilled in the art that according to the type of an organism used for the cell free extract solution, a TATA box (e.g., TATA(A/T)A(A/T)), or a Kozak consensus sequence (e.g., ACCATGG) upstream and downstream of an initiation codon ATG, is optionally selected.

As described above, since DNA lacks a termination codon in the first cassette, mRNA as a product of transcription and a peptide as a product of translation are continued to be coupled with a ribosome, resulting in a PRM complex. Further, by increasing the magnesium concentration, the peptide-ribosome-mRNA complex can be stabilized.

Thereafter, the step of selecting a PRM complex capable of specifically binding to an immobilized membrane model from the above-described PRM complexes is carried out. Specifically, for example, the remaining DNA is decomposed by DNaseI treatment. Thereafter, by increasing the magnesium concentration, the stability of the PRM complex is enhanced. The PRM complex is allowed to react with the immobilized membrane model for 1 to 60 min.

The immobilized membrane model may be obtained by immobilizing onto a solid phase liposomes having an artificial lipid bilayer imitating the cell membrane structures of various organisms depending on the purpose. Examples of a composition of an artificial lipid bilayer imitating the cell membrane structures of various organisms include those comprising acid phospholipids, such as phosphatidyl glycerol, phosphatidyl serine, and the like, but not cholesterol, in the case of the microorganism membrane model. Examples of an animal cell membrane model imitating the cell membrane structure of a healthy animal include those comprising cholesterol but not acid phospholipid. Specifically, for example, a microorganism membrane model comprises phosphatidyl choline and phosphatidyl glycerol having a ratio of 1:1 (including biotinylated phosphatidyl ethanolamine accounting for 1.4% of the total amount of phospholipid amount). For example, an animal cell membrane model comprises phosphatidyl choline, phosphatidyl glycerol, and cholesterol having a ratio of 10:1:1 (including biotinylated phosphatidyl ethanolamine accounting for 1.4% of the total amount of phospholipid amount). The present invention is not limited to the above-described examples. Either of the immobilized membrane models may contain a small amount of biotinylated phosphatidyl ethanolamine. The immobilized membrane model may be immobilized via biotin onto beads or plate coated with streptavidin.

A liposome having an artificial lipid bilayer may be any of MLV, LUV and SUV and may be preferably SUV having a diameter of 100 nm or less. SUV is prepared, for example, as follows. The above-described composition of phospholipids which will imitate the cell membrane structure of an organism is dissolved in chloroform solution and is dried in the presence of nitrogen gas, followed by substantially complete evaporation of an organic solvent overnight under reduced pressure, resulting in a phospholipid film. TBS (20 mM Tris-HCl, 150 mM NaCl (pH 7.6)) is added to the phospholipid film, followed by vigorous agitation, resulting in MLV. In addition, after LUV is prepared with ultrasonic treatment of MLV, SUV can be obtained by allowing the LUV to pass through a filter of a polycarbonate.

Immobilization to a solid phase is carried out by immobilizing a liposome comprising the above-described artificial lipid bilayer onto a solid phase, such as beads, a plate, or the like. When a liposome comprises a small amount of biotinylated phosphatidyl ethanolamine, the liposome can be immobilized on magnetic beads coated with streptavidin via biotinylated phosphatidyl ethanolamine. Note that when a liposome is immobilized on magnetic beads, a peptide specifically binding to an immobilized membrane model can be efficiently recovered since the beads can be recovered using a magnet.

Thereafter, reverse-transcription of mRNA in the selected PRM complex to DNA is carried out. Specifically, for example, the PRM complex specifically binding to an immobilized membrane model is collected together with the magnetic beads using a magnet, followed by washing with TBS (20 mM Tris-HCl, 150 mM NaCl (pH 7.6)). Only mRNA is allowed to elute with a buffer solution containing EDTA, followed by further purification. Thereafter, one cycle of RT-PCR is carried out to obtain a DNA library (e.g., C. thermo. polymerase one-step RT-PCR kit (manufactured by Roche)).

The screening comprising the above-described steps is carried out for at least 4 cycles, preferably at least 5 cycles, and more preferably at least 6 cycles. As a result, a peptide capable of specifically acting on a desired membrane structure can be preferably concentrated. Note that it is possible to use a well-known method, such as agarose electrophoresis to confirm whether or not DNA has been obtained after each cycle of screening.

After the final cycle of the screening, the resultant DNA is sequenced to determine the base sequence, and a corresponding amino acid sequence is determined. This procedure is well known. Specifically, for example, immediately after RT-PCR, the resultant DNA is inserted into a cloning vector by TA cloning or the like to determine the base sequence. Thereafter, a corresponding amino acid sequence is determined. Thus, an amino acid sequence of a peptide capable of specifically acting on a desired membrane structure can be known.

Note that it is preferable to carry out “prescreening” before screening in order to remove complexes in which the full length of a peptide library is not expressed due to errors in DNA synthesis or PCR. With this prescreening process, it is possible to significantly reduce the probability that incomplete base sequences is brought into an actual screening system.

In another aspect, prescreening is carried out, instead of the immobilized membrane model, using beads or a plate, onto which antibodies to a tag such as a heterologous epitope (e.g., FLAG, myc, the SV40 T antigen, glutathione S-transferase, 6 histidine (6His), maltose binding protein) are immobilized. For example, it is now assumed that anti-FLAG antibodies are employed. When the above-described library lacking a termination codon is subjected to transcription and translation using a cell free extract solution of E. coli, a plant, or an animal, clones having a frame shift or a termination codon are partially generated. A base sequence having a frame shift cannot correctly express the FLAG tag. Therefore, PRM complexes obtained by transcription and translation cannot be bound to the anti-FLAG antibodies, and cannot be recovered by a magnet, remaining in supernatant. Thereafter, the template DNA is decomposed by DNaseI treatment. The magnesium concentration is increased to enhance the stability of the PRM complex, which is in turn allowed to react with magnetic beads or a plate coated with anti-FLAG antibodies. Only complexes correctly expressing FLAG are adsorbed onto the beads or plate. Peptide-mRNA complexes correctly expressing the full length peptide and the magnetic beads are washed with TBS. EDTA is added to the complexes to elute only mRNA. The eluted mRNA is purified, followed by RT-PCR to obtain a library of DNAs having the full length. DNA having a non-full length base sequence is removed. Thereafter, the above-described screening (i.e., main screening) is carried out. Note that in prescreening, anti-myc antibodies, anti-His antibodies, or the like may be employed depending on the type of epitope tag introduced into a cassette, in addition to anti-FLAG antibodies.

When the present inventors actually carried out the method of the present invention using mast21, mast21 was selectively concentrated only if the microorganism membrane model was used. When mastoparanX was used instead of mast21, mastoparanX was screened for in both of the immobilized membrane models, i.e., the microorganism membrane model and the animal cell membrane model. Therefore, the method of the present invention is effective in screening for a peptide capable of distinguishing cell membrane structures. Note that the method of the present invention can be applicable to peptides capable of acting on a cell membrane structure in addition to mast21 and mastoparanX, such as magainins, tachyplesins, defensins, cecropins, PGLa, dermaseptins, and the like.

The method of the present invention was also applied to a library consisting of peptides expected to have potent antibacterial activity, such as tachyplesin and derivatives thereof, from which only amino acid sequences having deletions have been obtained by phage display. In this case, the full-length peptide could be efficiently obtained. Therefore, it was demonstrated that any influence of a peptide on organism during screening can be removed by carrying out all processes of random gene sequence expression and screening in a cell-free system without using organisms, such as microorganisms, phages, or the like. In other words, it is possible to significantly reduce the possibility that amino acid sequences expected to act on organisms are partially deleted or removed. Further, even when the method of the present invention was actually applied to a random peptide library, a peptide containing a specific amino acid sequence could be concentrated. Therefore, the screening method of the present invention is expected to be used as a method for efficiently screening for a peptide capable of acting on a biological membrane.

Liposomes used as a microorganism membrane model, in which acid phospholipids are exposed toward the outside of cells, can be used as a model of a cell having an abnormality (e.g., apoptotic cells, cancer cells) compared to healthy cells. Therefore, the screening method of the present invention is not limited to use in obtaining a novel peptide capable of specifically acting on the membrane of microorganisms. However, it will be understood by those skilled in the art that the screening method of the present invention may be used to obtain a peptide capable of specifically acting on cancer cells and/or apoptotic cells.

The screening method of the present invention was used to screen for a base sequence encoding the above-described peptide consisting of 21 random amino acids and the base sequence was determined. As a result, the present inventors found that a motif consisting of a basic amino acid K (lysine) or R (arginine) and several hydrophobic amino acids highly frequently appears in the obtained peptide. The basicity of a peptide is essentially required for the peptide to specifically act on the membrane of a microorganism negatively charged. An amphipathic helix and/or amphipathic β sheet structures increase the permeability of the membrane of microorganisms, impart pore forming capability, and damage the membrane of microorganisms. We found that a repeat of the above-described motifs imparts the basicity and the amphipathic helix and/or amphipathic β sheet structures, and completed the present invention.

The present invention provides a novel peptide capable of specifically acting on a biological membrane which is screened for by the screening method of the present invention. In one aspect, the biological membrane may be the membrane of a microorganism. Preferably, the microorganism maybe a pathogenic microorganism. In another aspect, the biological membrane may be the membrane of a eukaryotic cell. Preferably, the eukaryotic cell may be a mammalian animal cell. More preferably, the mammalian animal cell may be a cancer cell or an apoptotic cell. In another aspect, the biological membrane may be either the membrane of a microorganism or the membrane of a eukaryotic cell.

In a preferred embodiment, the peptide of the present invention comprises a sequence imparting an amphipathic helix and/or amphipathic β sheet structures, wherein the sequence may be preferably Z¹X¹X²X³Z²X⁴X⁵Z³X⁶X⁷X⁸Z⁴X⁹ or X¹Z¹X¹X¹X¹Z¹X¹X¹Z³X⁶X⁷X⁸Z⁴, where X¹ to X⁹ are any amino acids, two, three or four of Z¹ to Z⁴ are basic amino acids. More preferably, these basic amino acids may be lysine (K) or arginine (R). Even more preferably, at least two, preferably at least 3, 4, 5, 6 or 7 of X¹ to X⁹ may be hydrophobic amino acids.

In a preferred embodiment, the peptide of the present invention may comprise a DNA sequence encoding ZXXXZXXZXXXZXX, where Z is lysine (K) or arginine (R) and X is any amino acid).

The present invention also provides a pharmaceutical composition comprising an effective amount of the peptide of the present invention within a pharmaceutical acceptable carrier. In a specific embodiment, the pharmaceutical composition comprising the peptide of the present invention may be administered using a liposome, a microparticle, or a microcapsule. To achieve the sustained-release of the peptide of the present invention, use of such a composition is effective.

The present invention also provides a pharmaceutical pack or kit comprising one or more containers filled with one or more ingredients of the pharmaceutical composition of the present invention.

The present invention also provides an antibody to a peptide obtained by the screening method of the present invention.

Hereinafter, the present invention will be described by way of illustrative examples in greater detail. Note that the following examples are for illustrative purposes only and are not intended to the scope of the present invention.

### EXAMPLES

### Example 1

Preparation of a First Cassette to a Third Cassette

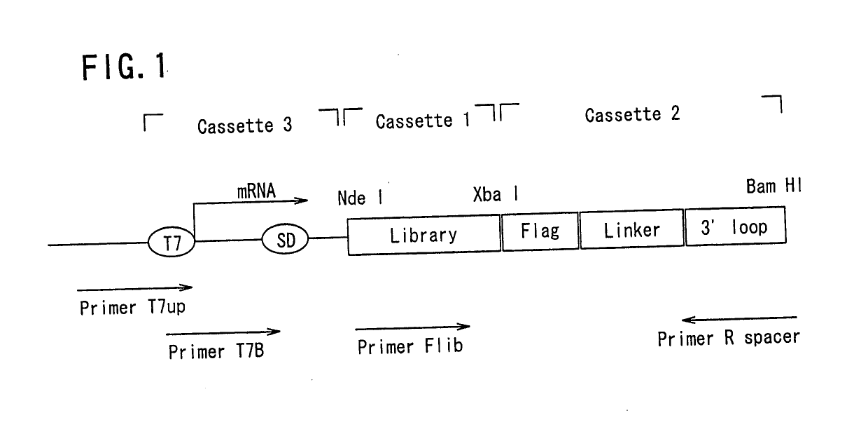

Referring to FIG. 1, genes for construction of DNA libraries were designed to comprise three portions (a first cassette, a second cassette, and a third cassette) in order to be applicable to the design of any library. The first cassette is a site into which a library of nucleic acid sequences are inserted, i.e., the base sequence of a gene corresponding to the library to be screened is inserted. Note that a base sequence to be inserted into the first cassette lacks a termination codon so that a peptide as a product of translation and mRNA as a product of transcription are continued to be coupled together via a ribosome in a PRM complex. In addition, a NdeI site and a XbaI site were added to a 5′ site and 3′ site of the first cassette, respectively, to ligate to the second cassette and the third cassette, respectively.

For the second cassette, a base sequence encoding a FLAG tag for prescreening was introduced into a 5′ site thereof, a 3′ stem-loop for increasing the stability of mRNA, and a linker sequence (a base sequence encoding four contiguous SEQ ID NO: 4×2) for allowing a translated peptide to move flexibly. The length of the linker sequence may be optionally extended where a unit length is four contiguous SEQ ID NO: 4×2. Further, an XbaI site is added to a 5′ site of the second cassette so as to ligate the second cassette with the first cassette.

The third cassette comprises all base sequences required for transcription and translation (a T7 promoter sequence (SEQ ID NO: 1), a Shine-Dalgarno (SD) sequence (SEQ ID NO: 2), and a 5′ stem-loop (SEQ ID NO: 3) for increasing the stability of post-transcriptional mRNA). The distance between the T7 promoter and the SD sequence was designed to be optimal. A restriction enzyme NdeI site was added to a 3′ site of the third cassette so as to ligate the third cassette with the first cassette.

### Example 2

Construction of DNA Libraries

Base sequences corresponding to a peptide library to be subjected to screening were chemically synthesized to prepare the first cassette. The base sequence used in the first cassette was (XXB)₂₀XAG and thus lacked a termination codon. In addition, a restriction enzyme site was added to a5′ site and3′ site of the first cassette. The first cassette and the second cassette were ligated together using DNA ligase. The ligation product of the first cassette and the second cassette was amplified using primers 3 and 4 (SEQ ID NO: 6 and SEQ ID NO: 7, respectively). The resultant PCR product was treated with NdeI, followed by purification. Thereafter, the product was ligated to the third cassette using DNA ligase. The resultant ligation product of the third cassette and the first and second cassettes was amplified using primers 1 and 4 (SEQ ID NO: 8 and SEQ ID NO: 7, respectively). The resultant PCR product was employed as a DNA library.

### Example 3

Transcription and Translation of DNA in Cell-Free System)

0.5 μg of the constructed DNA library was used in a one-step transcription/translation system having a total volume of 20 μl. The reaction system contained E. coli S30 extract solution and twenty amino acids. 0.8 μl of T7 RNA polymerase was added to the reaction system which was in turn allowed to undergo a reaction at 37° C. for 30 min. Thereafter, RNase-free DNaseI was added to the system, followed by a further reaction for 20 min to completely decompose template DNA. Thereafter, the system was cooled on ice while adding 1 M magnesium acetate solution to a final concentration of 50mM, thereby stabilizing a peptide-ribosome-mRNA complex (PRM complex).

### Example 4

Prescreening

Next, prescreening was carried out. With the prescreening process, complexes in which the full-length expression of a peptide of interest was prevented by a termination codon generated due to error in DNA synthesis or PCR, can be removed. Therefore, the probability that incomplete base sequences are brought into an actual screening system can be significantly reduced.

Biotinylated anti-FLAG antibodies were immobilized on magnetic beads coated with streptavidin, followed by a reaction with PRM complex solution for 1 h. DNA having a frame shift or an interrupting termination codon cannot correctly express the FLAG tag, and therefore, cannot be bound to anti-FLAG antibodies and cannot be recovered by a magnet, remaining in supernatant.

The beads recovered by a magnet were washed with cold TBS (20 ml Tris-HCl, 150 mM NaCl (pH 7.6)) containing 50 mM magnesium acetate 5 times. Thereafter, the beads were reacted with 20 mM EDTA solution on ice for 10 min to elute mRNA from the complex. The eluted mRNA was purified using a G-25 microspin column (manufactured by Amersham), followed by reverse-transcription and amplification by RT-PCR using primers 2 and 4 into DNA. The DNA was purified and was subjected to the following screening cycle.

### Example 5

Preparation of Immobilized Membrane Model

The microorganism membrane model employed was a liposome comprising phosphatidyl choline and phosphatidyl glycerol having a ratio of 1:1 (including biotinylated phosphatidyl ethanolamine accounting for 1.4% of the total amount of phospholipid amount). The animal cell membrane model employed was a liposome comprising phosphatidyl choline, phosphatidyl glycerol, and cholesterol having a ratio of 10:1:1 (including biotinylated phosphatidyl ethanolamine accounting for 1.4% of the total amount of phospholipid amount).

Chloroform solution containing a total weight of 20 mg of phospholipids was dried in the presence of nitrogen gas in a 15 mm-diameter glass test tube, and was then placed under reduced pressure overnight for complete evaporation of the organic solvent, resulting in a phospholipid film. TBS (20 mM Tris-HCl, 150 mM NaCl (pH 7.6)) was added to the phospholipid film, followed by vigorous agitation, resulting in MLV. The MLV were subjected to ultrasonication to produce LUV. The LUV were passed through polycarbonate filters having a pore size of 600 nm, 400 nm, and 100 nm, 10 times for each. As a result, SUV having a diameter of 100 nm or less were prepared. Magnetic beads coated with 100 μl (1 mg) of streptavidin (Roche) were washed with TBS and were suspended in 500 μl of TBS. Biotinylated liposomes having a phospholipid content of 16.8 μg were added to the suspension, followed by a reaction at 4° C. for 1 h while stirring, so that the biotinylated liposomes were immobilized on the magnetic beads. After reaction, the beads were washed with TBS and were stored at 4° C.

### Example 6

Screening of DNA Library Using Immobilized Membrane Model

Screening was carried out in accordance with the scheme as shown in FIG. 2. 0.5 μg of DNA library after prescreening was employed in a one-step transcription/translation system having a total volume of 20 μl, so that a peptide-ribosome-mRNA complex (PRM complex) was formed, as in Example 3.

Thereafter, the PRM complex was reacted with magnetic beads on which a biotinylated liposome had been immobilized (a microorganism membrane model liposome or an animal cell membrane model liposome) prepared in Example 5. The magnetic beads were recovered by a magnet, followed by washing with cold TBS containing 50 mM magnesium acetate, 5 times. Thereafter, the beads were reacted with 20 mM EDTA solution on ice for 10 min to elute mRNA from the complex. The eluted mRNA was purified using G-25 microspin column (manufactured by Amersham), followed by reverse-transcription and amplification by RT-PCR using primers 2 and 4 (SEQ ID NO: 9 and SEQ ID NO: 7, respectively). After RT-PCR, the amplification product was further purified and was subjected to additional screening.

RT-PCR was carried out using a C. thermo. polymerase one-step RT-PCR kit (manufactured by Roche). 0.3 pmol (final concentration) primer 2 (SEQ ID NO: 9), primer 4 (SEQ ID NO: 7), and 10 pg to 2 ng of mRNA were added to a reaction solution containing 0.4 mM dNTP, 5% DMSO, 0.5% DTT, and 0.8 unit RNase inhibitor (final concentration for each), where the total volume of the reaction solution was 25 μl. The reaction solution was subjected to heat treatment at 60° C. for 30 min and at 95° C. for 5 min using a thermal cycler, followed by 25 cycles of 95° C. for 30 sec, 57° C. for 30 sec, and 72° C. for 1 min, and further 72° C. for 6 min. Thus, the reverse-transcription reaction and the following DNA amplification were successively carried out. After RT-PCR, the resultant amplification product was further purified and was subjected to additional screening.

In this manner, screening was carried out in a total of 6 cycles to concentrate a peptide capable of specifically acting on a cell membrane. After RT-PCR, TA cloning was carried out and base sequencing was carried out. As a result, information about the amino acid sequence of a peptide capable of specifically acting on a desired membrane structure was obtained.

### Comparative Example

Screening Having a Combination of Phage Display and Immobilized Membrane Model System

In order to compare with the method of the present invention, screening was carried out in which phage display was combined with an immobilized membrane model system. Phage display was carried out using a T7 phage system (manufactured by Novagen) under the following conditions and procedures. For a gene encoding a peptide to be expressed, 5′-aatt was introduced into a 5′ site of one strand of a vector arm while 5′-agct was introduced into a 5′ site of the complementary strand of the vector arm in the case of chemical synthesis. Thereafter, double-stranded DNA was formed by annealing. The DNA was inserted into the left arm and right arm of the T7 phage vector. Prior to this insertion, the left and right arms had been treated to produce ttaa and agct overhangs, respectively. The left arm, inserted DNA, and the right arm were ligated using DNA ligase to construct an expression vector.

Thereafter, phage particles were formed by in vitro packaging. The in vitro packaging was carried out by adding 1 μg of DNA to 25 μL of T7 packaging extract solution, followed by a reaction at 22° C. for 2 h. The reaction was terminated by adding 270 μl of LB medium (10 g of Bacto tryptone, 0.5 g of Yeast Extract, 10 g of NaCl/L). The solution was employed in the next step within 24 h. When the solution would be employed after 24 h and within 1 week, 20 μL of chloroform was added to the solution which was in turn stored at 4° C.

The phage titer of the solution was determined. E. coli strain BL21 which is a host for the phage was used in the amplification of a phage library by a plate method. Specifically, 0.5 ml of BL21 which had been cultured overnight was added to 50 ml of fresh TB medium (12 g of Bacto tryptone, 2 g of Yeast Extract, 4 ml of glycerol, 100 ml of calcium phosphate/L), followed by culturing until the turbidity (OD600) reached 0.6 to 1.0. 1×10⁶ plaque forming units (pfu) of phage were added to 10 ml E. coli culture solution. 1 ml of aliquot was transferred to a 15 ml tube. Top agarose (1 g of Bacto tryptone, 0.5 g of Yeast Extract, 0.5 g of NaCl, 0.6 g of agarose) cooled to 45 to 50° C. was added to the tube, immediately followed by mixing and uniformly spreading the mixture onto a 15 mm-diameter LB plate. After the top agarose became solid, culture was continued at 37° C. until the entire plate was covered with phage plaques (3 to 4 hours). Thereafter, to elute the phage, 10 ml of elution buffer I (20 mM Tris-HCl (pH 8.0), 100 mM NaCl, 6 mM MgSO₄) was poured onto the plate. The plate was allowed to stand at 4° C. for 2 h to overnight. Thereafter, the phage was recovered into the elution buffer. The elution buffer was centrifuged at 3,000×g for 5 min. The resultant supernatant was used as a phage library. The phage titer was determined again. The phage library was stored at 4° C.

The thus-obtained phage library was used for screening as in Example 6. The phage library was added to magnetic beads on which a membrane model had been immobilized, followed by a reaction at 4° C. for 10 to 60 min. The beads having adsorbed phages were recovered by a magnet, followed by washing with TBS 5 to 10 times. Thereafter, elution buffer II (5 M NaCl, 1% SDS) was added to the beads, which were in turn allowed to stand for 20 to 30 min at room temperature. As a result, the phages adsorbed on the immobilized membrane model were eluted and recovered.

The recovered phage was multiplied by the above-described plate method, and was subjected to the next cycle of screening in which the phage would be adsorbed onto an immobilized membrane model. This process was carried out 4 times or more. When the phage titer was sufficiently increased, plaques which were separated at a sufficient distance from each other were recovered from each agarose. After recovery of phage DNA, the amino acid sequence of the absorbed peptide was sequenced.

### Example 7

Effect of Concentration of Mast21 Capable of Acting on the Membrane of Microorganisms

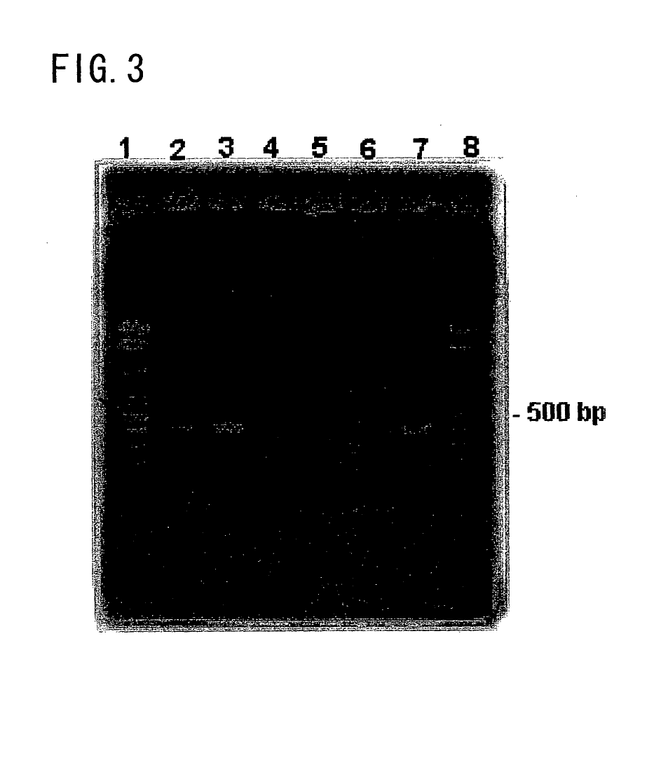

The final amplification product obtained in Example 6 was subjected to agarose gel electrophoresis. The result is shown in FIG. 3. In FIG. 3, lanes 1 and 8 indicate molecular weight markers. Lanes 2 to 4 indicate the results when a microorganism membrane model was used. Lanes 5 to 7 indicate the results when an animal cell membrane model was used. Lanes 2 and 6 indicate the results of mast21. Lanes 3 and 7 indicate the results of mastoparanX. Lanes 4 and 5 indicate the results when no library was inserted. MastoparanX was successfully screened for in both of the immobilized membrane models, so that DNA bands were detected at their appropriate molecular weight positions. In contrast, mast21 was successfully screened for only when the microorganism membrane model was used. In either case, the base sequence of the peptide was confirmed to have its full length without a deletion or the like.

The base sequence of a gene encoding mast21 (SEQ ID NO: 5) was inserted into the first cassette and the above-described screening was carried out. Only when a microorganism membrane model was used, a signal was obtained from the corresponding DNA after RT-PCR. The screening result was in agreement with the property of mast21 that mast21 specifically acts on the membrane of microorganisms but not the membrane of animal cells. Even when the base sequence was determined after repetition of cycles, substantially all clones encoded the full-length sequence and only a few clones had deletions. This means that the method of the present invention is effective in screening of a peptide capable of specifically acting on the membrane of microorganisms. When a screening system having a combination of phage display and an immobilized membrane model system was employed, the efficiency of concentration of the base sequence of a peptide was considerably poor and the obtained base sequence always had deletion mutations. This indicates that the method of the present invention allows screening even when the subject of screening is a peptide capable of affecting the growth of microorganisms.

### Example 8

Effect of Concentration of MastoparanX Nonspecific Action

Instead of the base sequence of a gene encoding mast21 (SEQ ID NO: 5), the base sequence of a gene encoding mastoparanX (SEQ ID NO: 10) was inserted into the first cassette and the above-described screening was carried out. In either the case of the microorganism membrane model or the animal cell membrane model, a signal was obtained from the corresponding DNA after RT-PCR (lane 3 and 6 in FIG. 3). This result is in agreement with the property of mastoparanX that it acts on all types of film, i.e., no selectivity. Even when the base sequence was determined after repetition of cycles, substantially all clones encoded the full-length sequence and only a few clones had deletions. This means that the method of the present invention is effective in screening of a peptide capable of acting on biological membranes, such as the membrane of microorganisms, the membrane of animal cells, and the like. When a screening system having a combination of phage display and an immobilized membrane model system was employed, the efficiency of concentration of the base sequence of a peptide was considerably poor and the obtained base sequence always had deletion mutations. This:indicates that the method of the present invention allows screening even when the subject of screening is a peptide capable of affecting the growth of organisms.

### Example 9

Result of Screening Library Expected to Have Potent Antibacterial Property

A library containing tachyplesin having potent antibacterial activity and derivatives thereof was subjected to the screening system of the present invention. The same library was subjected to a screening system having a combination of phage display and an immobilized membrane model system. As a result of the latter, an increase in phage titer was considerably poor, and the eventually obtained amino acid sequences had deletion mutations at a frequency of 100%. In contrast, the result of the screening method of the present invention shows that the full-length peptide was efficiently obtained. Thus, it was demonstrated that the method of the present invention is adaptable to a peptide lethal to organisms or capable of affecting the growth of organisms.

### Example 10

Screening of a Novel Peptide Capable of Specifically Acting on Microorganism Membranes Using a Library Encoding Random Peptides

In the first cassette, base sequences encoding a peptide consisting of 21 random amino acids were employed. Note that if the amino acid is perfectly random, a termination codon occurs, which prevents generation of a full-length peptide: and therefore, base sequences, (XXB)₂₀XAG, were used, where X represents A, T, G or C, and B represents T or G. By limiting the third codon to T or G, the occurrence of a possible termination codon was removed. For the purpose of ligating the cassettes, a base sequence encoding the 21^{st} amino acid needed to be XAG. Therefore, possible amino acids were limited to Tyr, His, Asn, and Asp. The random peptide library was used as the first cassette and was ligated with the other cassettes in the same manner as that described in Example 6.

The PCR final product obtained by the screening method in Example 6 was purified, followed by TA cloning using a TOPO TA cloning kit (manufactured by Invitrogen) to introduce a plasmid into a host TOPO10. Colonies having introduced plasmids containing the inserted peptide sequence were selected from TOPO10 colonies grown on a plate. Plasmids were extracted from each TOPO10. The resultant 50 clones were each sequenced with ABI PRISM310 (manufactured by PE Applied Biosystems). Among the 50 clones, 48 clones encoded 21 amino acids without a deletion or the like. Such a result cannot be obtained by phage display or the like. The resultant sequences are shown in Table 1.

Table 1. Random peptides capable of acting on membrane structure, concentrated from libraries by the screening method of the present invention

| Name No | | SEQ ID NO. |
|---|---|---|
| 1. | VWAWVFGASTRERARVGWQCY | (11) |
| 2. | CFVLPGVRPCSSHILTLSFSY | (12) |
| 3. | EGVRDMFRRCLWISLRSWCVH | (13) |
| 4. | GGVYASCASYLLALLSRVGGN | (14) |
| 5. | SDSASVSRVGGLWPTCCPH | (15) |
| 6. | WGRVDNSGSWGRVGAPWRYLH | (16) |
| 7. | AAVYLVTSLFGIVTGVREDH | (17) |
| 8. | DSRGVRAFACDYVLFVLWVPY | (18) |
| 9. | DLAPVRVGFYNAYRELRVFRY | (19) |
| 10. | RVGALYYFMVWYLMWFFLLFH | (20) |
| 11. | PVLDAGSVYLGYLGVRFLSY | (21) |
| 12. | VRNRVIARVGGVPYVGGPCYN | (22) |
| 13. | ACCLVRFYSHGRGKRVGFLWY | (23) |
| 14. | LRYSGLLGFPLWVGRIFVCVD | (24) |
| 15. | RMRCVSLELVVYGGGVRMWEN | (25) |
| 16. | FMGYGRSVWVVSSSLVLCIYD | (26) |
| 17. | ARMLWGRGTTLLLIRRRVSAY | (27) |
| 18. | VDLSWYASCRVSICVFVVVY | (28) |
| 19. | WPNYQSREHMRVSSRMYYYFY | (29) |
| 20. | CVVRVSNVKAAALIPGVVSRH | (30) |
| 21. | WWCLLGYWALGGNHSAKVSSY | (31) |
| 22. | YGSYLEALWWGTSACWALRY | (32) |
| 23. | GVAVDCAVVGWALRVLGVHSY | (33) |
| 24. | RCLEAGKIWWGALRSHLAVYD | (34) |
| 25. | GSGSAVGWALRSYASGLAIAY | (35) |
| 26. | HAWARWMGWGHGGVLSWALRY | (36) |
| 27. | FVSWALRYSRCLVWLCWFPNY | (37) |
| 28. | VKGNPVFDHRHFSLWGALREY | (38) |
| 29. | FVQHWSFTAGSRSDRAPYPGH | (39) |
| 30. | AGWVNARRMWSLMPLMWLWSY | (40) |
| 31. | DRTTGRWFYIRRTAEVLGWTY | (41) |
| 32. | RFINPTSHCFGSLSLWRQLSY | (42) |
| 33. | VGCLVSVGSVWGCSSVVVRVY | (43) |
| 34. | RMESGAPLAAYGKMRLRPGTH | (44) |
| 35. | VWNRVIARCGGVLYVGGPCTN | (45) |
| 36. | SGHMHSYWPTTWILVLIRRTY | (46) |
| 37. | LEVLVWYSLWSYWLDVAAASH | (47) |
| 38. | SWGGGFYDWSYVGGGAYWAY | (48) |
| 39. | MGLFRSYKYRFVHDSESSFN | (49) |
| 40. | MALYLAWYGCSDSAVVMLADD | (50) |
| 41. | MYCWRMLANSCAARMVLAMRN | (51) |
| 42. | VIVNVAVLYRRCWPCAEFWPY | (52) |
| 43. | RLGSFYPLLWRLVSHEYSLWH | (53) |
| 44. | RYWFGRWRCFYGPFVSSYFLY | (54) |
| 45. | VCCCRCLPWSYMCEWGSMRLY | (55) |
| 46. | VLKIHSWHNWVYGVMLYDMEY | (56) |
| 47. | MGYAWDLGLRMGPYFLMDLIN | (57) |
| 48. | SDKCAPVCYVMDRLCLANWD | (58) |

### Example 11

Screening of a Novel Peptide Capable of Specifically Acting on Microorganism Membranes Using a Library Encoding Semirandom Peptides

The membrane of microorganisms is negatively charged since acidic lipids (PG, PS, lipopolysaccharide, and the like) are exposed on the surface layer thereof. Therefore, the function of a peptide capable of specifically acting on the membrane of microorganisms essentially requires the basicity of the function. It is believed that host defense peptides have an amphipathic structure which increases the permeability thereto of the membrane of microorganisms, imparts pore forming capability, and damages the membrane of microorganisms.

Motifs consisting of several specific amino acids (e.g., RVG, ALR, RVS, etc.) highly frequently occurred in the peptides obtained in Example 10. These motifs consisted of a basic amino acid K (lysine) or R (arginine) and several hydrophobic amino acids. These motifs are believed to be the minimum unit essentially required for linkage to the membrane of microorganisms. We expected that a repeat of these motifs would impart an amphipathic helix or amphipathic P sheet structure. Therefore, we designed a library in an attempt to obtain a peptide capable of more effectively binding to the membrane of microorganisms and subjected the peptide to screening. Specifically, a library of DNA sequences encoding 14 amino acids, ZXXXZXXZXXXZXX (Z represents K (lysine) or R (arginine) and X represents any amino acid), in which K or R was placed at a position which will provide an amphipathic peptide, were subjected to screening. The same procedure as that described in Example 10 was carried out, except for the peptide library.

Screening was carried out in accordance with the method described in Example 6. In the final step, the reverse-transcription product from the resultant mRNA was subjected to TA cloning and base sequencing as in Example 10. Thereafter, the corresponding amino acid sequence was determined. 50 clones were subjected to sequencing. Among them, 47 clones perfectly encoded the full length without a deletion or the like. The obtained sequences are shown in Table 2. As shown in Table 2, sequences, such as KVZ, RVZ, KSV, KV, and RSV, are seen in a plurality of the obtained peptides. Thus, in most of the peptides, K/R is followed by a hydrophobic amino acid, rarely a hydrophilic but nonpolar amino acid, such as S (serine) or the like, but not a basic amino acid or an acidic amino acid. This supports our expectation that peptides obtained by the screening method of the present invention are likely to have an amphipathic helix or amphipathic 5 sheet structure and is useful as a host defense peptide.

Table 2. Random peptides capable of acting on membrane structure, concentrated from semi random libraries by the screening method of the present invention

| Name No | | SEQ ID NO. |
|---|---|---|
| 1. | RPVFRTYRSVVKSG | (59) |
| 2. | NRACLKRPRYLRKH | (60) |
| 3. | KACVRFSKSTSKRY | (61) |
| 4. | RFRPKAVRYRIKFN | (52) |
| 5. | RSHFRRVKRHSKIP | (63) |
| 6. | KDVLRNHKHSDRVG | (64) |
| 7. | KSARKVLKLYRKIT | (65) |
| 8. | KKNSCRDGRFSRKC | (66) |
| 9. | KGYFRGRRSYLRAF | (67) |
| 10. | KGCAKVLKRITRHI | (68) |
| 11. | KGRHRHCRYILRGN | (69) |
| 12. | KCTFRRRRLIIKPS | (70) |
| 13. | KTDWFRVLMTFLMD | (71) |
| 14. | RGFVRLIKPYAEAS | (72) |
| 15. | RNLCRSLRSHLEA | (73) |
| 16. | KIPGRFTRAGRKTT | (74) |
| 17. | KSDHRVAKNLPKTI | (75) |
| 18. | RRTGRIDKVSVKAY | (76) |
| 19. | KVLIKLAKCCIRIS | (77) |
| 20. | RAACRDSKLCSRYY | (78) |
| 21. | KHFVRCPKCAVRSS | (79) |
| 22. | KISDRNSKHHCRSS | (80) |
| 23. | KVGLIVDKASVKTA | (81) |
| 24. | RDVCKSSRHSHKGS | (82) |
| 25. | RFVSKGTDAINRRS | (83) |
| 26. | KGNCRLYRLRCKVV | (84) |
| 27. | RLLLKAVRFCCKCF | (85) |
| 28. | KGGGKVGKHTRSR | (86) |
| 29. | RHFRKNCKFCHRHC | (87) |
| 30. | KRCTKVYRAYTKLT | (88) |
| 31. | KSYGKAPKFVGRIC | (89) |
| 32. | RAAIRHFRSATKRP | (90) |
| 33. | KYSARFCKYGGRSH | (91) |
| 34. | RFTARVRKSVFRSC | (92) |
| 35. | KVYSRSSKSAHKCF | (93) |
| 36. | KRAYKDARHIYLCS | (94) |
| 37. | KIFVRTIRAAHKRD | (95) |
| 38. | KGGGKVGKHTRSR | (96) |
| 39. | KSLTKCCKVLRLSC | (97) |
| 40. | RCDIKSVKHILRCS | (98) |
| 41. | KASVRNSKNLPRFC | (99) |
| 42. | KGARFLAKHLIRHY | (100) |
| 43. | RHVPKANKGADRSC | (101) |
| 44. | KTSWVRAAALVVVH | (102) |
| 45. | KSVNKDVRISLRD | (103) |
| 46. | KCIARRGRLPVKRY | (104) |
| 47. | KVLFRHARSSCKHY | (105) |

### Example 12

Mast21R, a variant of mast21

In the amino acid sequence of mast21 (SEQ ID NO: 5) capable of specifically acting on the membrane of microorganisms but not the membrane of animal cells, K (lysine) was substituted with R (arginine). The resultant peptide was designated mast21R (SEQ ID NO: 107). Mast21R is capable of specifically acting on the membrane of microorganisms and has potent antibacterial activity, similar to mast21.

Table 3. Mast21 variants maintaining the capability of specifically acting on a membrane structure and high antibacterial activity of mast21

| Name No | | SEQ ID NO. |
|---|---|---|
| mast21 | KNWKGIAGMAKKLLGKNWKLM | (5) |
| mast21N | KNWKGIAGMAKKLLGKNWKLM-NH2 | |
| mastWR | KNRKGIAGMAKKLLGNKWKLM | (106) |
| mast21R | RNWRGIAGMARRLLGRNWRLM | (107) |

### Example 13

Evaluation of the Membrane Specificity of a Novel Peptide Using Immobilized Membrane Models

Synthesis of peptides was outsourced to Sawady Technology (Tokyo). After chemical synthesis, the peptide was purified to 95% or more by HPLC. The molecular weight of the purified peptide was determined by mass spectrometry.

The ability to act on a cell membrane structure was evaluated using a partially modified version of the method described in JP No. 2967925 using a fluorescent substance (calcein) containing model membrane (liposome).

(A: Method for Preparing a Fluorescent Substance (Calcein) Containing Model Membrane (Liposome))

Three model membranes having a different phospholipid ratio were prepared: a typical microorganism membrane model, liposome A (PC/PG=1/1); a model close to the microorganism membrane model, liposome B (PC/PG=10/1); and a typical healthy animal cell membrane model, liposome C (PC/PG/Ch1=10/1/1), where PC represents phosphatidyl choline, PG represents phosphatidyl glycerol, and Ch1 represents cholesterol.

The phospholipids having the above-described ratio were prepared in chloroform solution (20 mg/ml) and transferred to a 15 mm-diameter test tube. The solution was concentrated while spraying nitrogen gas, resulting in formation of a lipid thin film on the inner wall of the test tube. The lipid thin film was placed under reduced pressure in a desiccator overnight, so that the organic solvent was completely evaporated. Thereafter, 1.5 ml of 10 mM HEPES solution (pH 7.4) containing 70 mM calcein was added to the lipid thin film, followed by agitating for 10 min with an agitator (manufactured by Vortex) to peel off the lipid thin film and form MLVs. Thereafter, the resultant suspension was subjected to ultrasonication until it became transparent, resulting in LUVs containing calcein. To separate the calcein containing liposome from free calcein, the solution was subjected to gel filtration (Sepharose CL-4B, 1 cm×25 cm, flow rate 0.25 ml/min, fraction size 2 ml). Fractions containing the calcein containing liposome were collected, and were subjected to the following experiment. The diameter of the liposome was several hundreds of nanometers. The liposome was quantified by determining the amount of the phospholipids using a phospholipid test wako (manufactured by Wako Pure Chemical Industries).

(B: Detection of the Action of a Peptide on Membranes)

Liposomes were added to 96-well microplates and were diluted with 10 mM HEPES buffer solution. 1, 2, 4, and 5 μM (final concentration) of aqueous peptide solution were added to 100 μl (20 μM)/well of the calcein containing liposome. Thereafter, at prescribed times, the amount of leaked calcein was detected using a microplate fluorometer (SPECTRAmax GEMINI manufactured by Molecular Devices) at an excitation wavelength of 485 nm and a fluorescence wavelength of 538 nm. Calcein does not emit fluorescence within a liposome due to fluorescence quenching. However, if a peptide acts on the liposome and forms pores in the liposome, calcein leaks out of the liposome. The leaked calcein emits fluorescence at the excitation wavelength.

The evaluation of the action of a peptide on membranes is represented by the relative fluorescence intensity. Specifically, TritonX-100 (final concentration: 1%) was added to the liposome so that the liposome was completely destroyed. The fluorescence intensity was regarded as 100% when all calcein leaked out of the liposome. The fluorescence intensity was 0% when only a buffer solution was added. The fluorescence intensity of 100% corresponds to a value of 5,000 or more. The result is shown in Table 4.

TABLE 4
Activity of peptides on a membrane structure

| | | | Liposome | | | | Liposome | | | | Liposome | | | |
|---|---|---|---|---|---|---|---|---|---|---|---|---|---|---|
| Name of | | | A (μM) | | | | B (μM) | | | | C (μM) | | | |
| peptide | Sequence/peptide concentration | | 1 | 2 | 4 | 5 | 1 | 2 | 4 | 5 | 1 | 2 | 4 | 5 |
| Ribo-1 | RLAWG | (SEQ ID NO:108) | nd | nd | nd | nd | nd | nd | nd | nd | nd | nd | nd | nd |
| Ribo-2 | GWALR | (SEQ ID NO:109) | nd | nd | nd | nd | nd | nd | nd | nd | nd | nd | nd | nd |
| RVL | RVL | (SEQ ID NO:110) | nd | nd | nd | nd | nd | nd | nd | nd | nd | nd | nd | nd |
| RVG | RVG | (SEQ ID NO:111) | nd | nd | nd | nd | nd | nd | nd | nd | nd | nd | nd | nd |
| KVG | KVG | (SEQ ID NO:112) | nd | nd | nd | nd | nd | nd | nd | nd | nd | nd | nd | nd |
| KVL | KVL | (SEQ ID NO:113) | nd | nd | nd | nd | nd | nd | nd | nd | nd | nd | nd | nd |
| KVL3 | KVLKVLKVL | (SEQ ID NO:114) | nd | nd | nd | nd | nd | nd | nd | nd | nd | nd | nd | nd |
| KVL+ | KVL ALRL | (SEQ ID NO:115) | nd | nd | nd | nd | nd | nd | nd | nd | nd | nd | nd | nd |
| KVL5 | KVLKVLKVLKVLKVL | (SEQ ID NO:116) | nd | 16 | 30 | 45 | 30 | 40 | 40 | 40 | 8.5 | 14 | 18 | 19 |
| *KVL-mastX | KVL INWKGIAAMAKKII | (SEQ ID NO:117) | 9 | 15 | 28 | 55 | 20 | 40 | 54 | 54 | 5.7 | 14 | 23 | 28 |
| *RVG-mastX | RVG INWKGIAAMAKKII | (SEQ ID NO:118) | nd | 9 | 25 | 30 | 10 | 26 | 38 | 42 | nd | nd | 6 | 8.5 |
| ALR | ALR | (SEQ ID NO:119) | nd | nd | nd | nd | nd | nd | nd | nd | nd | nd | nd | nd |
| ALR5 | ALRALRALRALRALR | (SEQ ID NO:120) | nd | 6 | 18 | 47 | nd | 28 | 35 | 46 | nd | 8 | 13 | 18 |
| mast21 | KNWKGIAGMAKKLLGKNWKLM | (SEQ ID NO:5) | 24 | 60 | 73 | 78 | 18 | 25 | 38 | 40 | 8 | 13 | 17 | 19 |
| mast21N | KNWKGIAGMAKKLLGKNWKLM-NH2 | | 25 | 68 | 84 | 84 | 20 | 60 | 78 | 86 | 28 | 48 | 56 | 61 |
| mastWR | KNRKGIAGMAKKLLGNKWKLM | (SEQ ID NO:106) | 8 | 15 | 23 | 28 | nd | 10 | 15 | 18 | nd | nd | 8 | 8 |
| mast21R | RNWRGIAGMARRLLGRNWRLM | (SEQ ID NO:107) | 13 | 22 | 39 | 56 | 12 | 26 | 38 | 40 | 8.5 | 8.5 | 8.5 | 8.5 |
| *mastoparanX | INWKGIAAMAKKLL | (SEQ ID NO:10) | 65 | 78 | 80 | 82 | 60 | 76 | 78 | 78 | 38 | 68 | 76 | 82 |

Leakage of fluorogens when a peptide was allowed to act on a cell membrane model (liposome) including fluorogen (calcein) is represented by % (the fluorescence strength was 100% when the liposome was completely destroyed with surfactant treatment and all calceins were leaked).
Liposome A: microorganism membrane model.
Liposome C: animal cell membrane model.
*KVL-mast, RVG-mast: mastoparan X with various sequences at the N terimus thereof. Mastoparan X has a high pore forming capability and lacks selectivity.

(C: Discussion)

For peptides of the minimum unit (3 amino acids) for acting on a cell membrane structure, substantially no significant action on a membrane was detected (nd: 5% or less). However, peptides obtained by tandemly linking these sequences expected to have the ability to specifically act on a cell membrane structure (e.g., KLV5: a repeat of 5 KLV sequences, KLVKLVKLVKLVKLV) were confirmed to have such an ability. Although not described in the Tables, the action of KLV and the like are enhanced with an increase in the chain length. KLV3 was observed to have the ability to act on a membrane structure which was increased by a factor of several percents as compared to KLV. This increase shows that KLV3 is capable of specifically acting on a cell membrane structure.

For example, KVL5 and ALR5 strongly acted on liposome A (a typical microorganism membrane model), but significantly weakly acted on liposome C (an animal cell membrane model).

MastoparanX acted on all of liposomes A, B and C to substantially the same level (data not shown). MastoparanX is a peptide whose antibacterial activity and cytotoxicity both are potent. When a peptide, such as KLV or the like, was added to this peptide, the action on liposome C was dramatically reduced, i.e., the specificity to the membrane of microorganisms was increased. This indicates that a peptide, such as KLV or the like, is a minimum unit which functions as a signal sequence capable of selectively acting on microorganism cells.

Mast21R obtained in Example 11 exhibited a potent and specific action on the microorganism membrane model. The action was significantly greater than that of mast21.

### Example 14

Evaluation of Antibacterial Activity

(A: Evaluation Method for Antibacterial Activity)

Antibacterial activity was evaluated in accordance with US National Committee for Clinical Laboratory Standard (NCCL Documents M7-A3). Specifically, the minimum concentration of a peptide capable of inhibiting the growth of bacteria was determined using microtiter plates. Bacteria were cultured for 16 h in sensitivity measurement broth medium (casamino acid: 16.5 g, beef heart extract: 3.0 g, soluble starch: 1.5 g, glucose: 2.0 g, L-tryptophan: 0.05 g, L-cystine: 0.05 g, biotin: 5 μg/L), followed by measurement of absorbance at A₆₀₀. According to the correlation between the previously obtained turbidity and the colony forming unit (CFU), each bacterial strain was diluted to a prescribed CFU with sensitivity measurement broth medium to a final concentration of 5×10⁵ CFU/ml. For each peptide, 5 mM aqueous solution and 1.6 mM solution with sensitivity measurement broth medium were prepared, and thereafter, were subjected to serial dilution (minimum: 0.78 μM). The peptide serial dilutions (50 μl for each) were added to respective wells of the microplate to which 50 μl of the bacterial solution had been added (the final peptide concentration ranged from 0.39 μM to 800 μM). Negative control did not contain the peptide. The plate was allowed to stand at 37° C. for 18 h for culture so that a minimum concentration which inhibits the bacterial growth (minimum inhibitory concentration: MIC) was determined. The result is shown in Table 5. The unit of concentration described in Table 5 is μM.

TABLE 5
Antibacterial activity of peptides

| | | | Strain/MIC (μM) | | | | | | | | |
|---|---|---|---|---|---|---|---|---|---|---|---|
| Name of | | | B. subtilis | B. sereus | S. aureus | S. aureus | E. coli | E. coli | E. coli | S. enteritidis | S. enteritidis |
| Peptide | Sequence | | IFO13722 | IFO3457 | IFO13276 | JCM2413 | JCM1649 | CR-3 | CE-273 | ATCC1891 | ATC14028 |
| Ribo-1 | RLAWG | (SEQ ID NO:108) | >800 | >800 | >800 | 800 | >800 | 800 | >800 | >800 | 800 |
| Ribo-2 | GWALR | (SEQ ID NO:109) | >800 | >800 | >800 | 800 | >800 | 800 | >800 | 800 | 800 |
| RVL | RVL | (SEQ ID NO:110) | >800 | >800 | >800 | >800 | >800 | 800 | >800 | >800 | 800 |
| RVG | RVG | (SEQ ID NO:111) | >800 | >800 | >800 | 800 | >800 | 800 | 800 | >800 | 800 |
| KVG | KVG | (SEQ ID NO:112) | >800 | >800 | >800 | 800 | >800 | 800 | 800 | >800 | 800 |
| KVL | KVL | (SEQ ID NO:113) | 800 | 800 | 800 | 400 | >800 | 800 | 800 | >800 | 800 |
| KVL3 | KVLKVLKVL | (SEQ ID NO:114) | >200 | >200 | >200 | >200 | >200 | >200 | >200 | >200 | >200 |
| KVL+ | KVL ALRL | (SEQ ID NO:115) | >200 | >200 | >200 | >200 | >200 | >200 | 3.1 | >200 | >200 |
| KVL5 | KVLKVLKVLKVLKVL | (SEQ ID N0:116) | 25 | 200 | 100 | 200 | 12.5 | 25 | 6.3 | 25 | 100 |
| KVL-mastX | KVL INWKGIAAMAKKII | (SEQ ID NO:117) | 25 | >200 | >200 | 50 | 100 | 50 | 25 | 100 | >200 |
| RVG-mastX | RVG INWKGIAAMAKKII | (SEQ ID NO:118) | 50 | >200 | >200 | 200 | >200 | 50 | 50 | 100 | 200 |
| ALR | ALR | (SEQ ID NO:119) | >200 | >200 | >200 | >200 | >200 | >200 | >200 | >200 | >200 |
| ALR5 | ALRALRALRALRALR | (SEQ ID NO:120) | 25 | 100 | 400 | 200 | 100 | | 100 | 100 | |
| *mast21 | KNWKGIAGMAKKLLGKNWKLM | (SEQ ID NO:5) | 6.3 | 3.1 | 25 | 12.5 | 6.3 | 6.3 | 3.1 | 6.3 | 6.3 |
| *mast21N | KNWKGIAGMAKKLLGKNWKLM-NH2 | | 3.1 | 3.1 | 12.5 | 12.5 | 3.1 | 3.1 | 3.1 | 6.3 | 6.3 |
| *mastWR | KNRKGIAGMAKKLLGNKWKLM | (SEQ ID NO:106) | 50 | 50 | 50 | 50 | 50 | 25 | 12.5 | 12.5 | 25 |
| *mast21R | RNWRGIAGMARRLLGRNWRLM | (SEQ ID NO:107) | 1.56 | 3.1 | 6.25 | 6.25 | 3.1 | 1.6 | 3.1 | 3.1 | 3.1 |
| mastoparan X | INWKGIAAMAKKLL | (SEQ ID NO:10) | 3.1 | 3.1 | 1.56 | 12.5 | 3.1 | 3.1 | 3.1 | 3.1 | 6.3 |

(B: Discussion)

Usually, if a substance has a MIC of 100 μg/ml or less, the substance is evaluated as a promising antibacterial agent. In the case of peptides, their concentrations vary greatly, depending on the molecular weight. For the sake of convenience, a peptide was herein judged to be promising with the MIC was 100 μM or less.

KLV5, ALR5, and the like, which specifically acted on the membrane of microorganisms were confirmed to have antibacterial activity. In addition, KLV6 (a repeat of 6 KLV sequences: KLVKLVKLVKLVKLVKLV) and ALR6 (a repeat of 6 ALR sequences: ALRALRALRALRALRALR) exhibited an increased level of activity compared to KLV5 and ALR5, respectively. Their antibacterial spectra extended from gram-negative bacteria, such as E. coli, to gram-positive bacteria, such as S. aureus.

The results for antibacterial activity were substantially in agreement with the results for the action on the membrane of microorganisms. Since the microorganisms used in the examples were infectious microorganisms as well as putrefactive microorganisms, the peptide of the present invention is useful for treatment of infectious diseases and/or prevention of putrefaction.

For mast21R, potent antibacterial activity and a broad antibacterial spectrum were observed, indicating that mast21R is a promising sequence as an antibacterial agent.

### Example 15

Evaluation of Hemolytic Property

(A: Method for Evaluating Hemolytic Property)

Action on erythrocytes was evaluated as a reference for action on the animal cell membrane model.

Fresh blood collected from a human was centrifuged at 700×g for 5 min. The resultant erythrocytes were washed in isotonic solution (0.15 mM NaCl/phosphate buffer solution (pH 7.4)) to prepare an erythrocyte solution. 80 μl of isotonic solution of each peptide was added to 720 μl of the erythrocyte solution (2×10⁷ cells/ml) (its final concentration is described in Table 6), followed by gentle mixing at 37° C. for 30 min for a reaction. After reaction, the mixture was centrifuged at 700×g for 5 min. Hemoglobin recovered in the supernatant was measured by determining the absorbance at 540 nm.

The hemolytic property is shown with relative values.

Specifically, the absorbance of only the isotonic solution is regarded as 0%, while the absorbance is regarded as 100% when the erythrocytes were substantially completely destroyed with TritonX-100. The result is shown in Table 6.

TABLE 6
Hemolytic property of peptides

| Name of | | | | | | |
|---|---|---|---|---|---|---|
| peptide | Sequence | | 1 μM | 5 μM | 10 μM | 100 μM |
| Ribo-1 | RLAWG | (SEQ ID NO:108) | nd | nd | nd | nd |
| Ribo-2 | GWALR | (SEQ ID NO:109) | nd | nd | nd | nd |
| RVL | RVL | (SEQ ID NO:110) | nd | nd | nd | nd |
| RVG | RVG | (SEQ ID NO:111) | nd | nd | nd | nd |
| KVG | KVG | (SEQ ID NO:112) | nd | nd | nd | nd |
| KVL | KVL | (SEQ ID NO:113) | nd | nd | nd | nd |
| KVL3 | KVLKVLKVL | (SEQ ID NO:114) | nd | nd | nd | nd |
| KVL+ | KVL ALRL | (SEQ ID NO:115) | nd | nd | nd | nd |
| KVL5 | KVLKVLKVLKVLKVL | (SEQ ID NO:116) | nd | nd | nd | nd |
| KVL-mastX | KVL INWKGIAAMAKKII | (SEQ ID NO:117) | nd | nd | nd | nd |
| RVG-mastX | RVG INWKGIAAMAKKII | (SEQ ID NO:118) | nd | nd | nd | nd |
| ALR | ALR | (SEQ ID NO:119) | nd | nd | nd | nd |
| ALR5 | ALRALRALRALRALR | (SEQ ID NO:120) | nd | nd | nd | nd |
| *mast21 | KNWKGIAGMAKKLLGKNWKLM | (SEQ ID NO:5) | nd | nd | nd | 5.4 |
| *mast21N | KNWKGIAGMAKKLLGKNWKLM-NH2 | | nd | nd | nd | 13 |
| *mastWR | KNRKGIAGMAKKLLGNKWKLM | (SEQ ID NO:106) | nd | nd | nd | nd |
| *mast21R | RNWRGIAGMARRLLGRNWRLM | (SEQ ID NO:107) | nd | nd | nd | 13 |
| **mastoparan X | INWKGIAAMAKKLL | (SEQ ID NO:10) | 5.4 | 18.8 | 86 | 93 |
| KVL6 | KVLKVLKVLKVLKVLKVL | (SEQ ID NO:122) | nd | nd | nd | nd |
| ALR6 | ALRALRALRALRALRALR | (SEQ ID NO:121) | nd | nd | nd | nd |

Hemolytic property is represented by a relative value where the value is 100 when erythrocytes were completely hemolysed.

(B: Discussion)

MastoparanX which is a peptide capable of acting on the membrane of animal cells and therefore having a high level of cytotoxicity exhibited a high level of hemolytic property. All of the obtained peptides exhibited a low level of hemolytic property. This indicates the validity of the method in which the sequence of a peptide incapable of acting on the animal cell membrane model is screened for. This result is in agreement with the result shown in Table 4 (the action on membranes) (a low level action on liposome C). In addition, the result indicates that the obtained peptides have a low level of action on the membrane of animal cells, including a human, and may function as highly safe antibacterial agents and may function as signal sequences capable of specifically acting on the membrane of microorganisms.

### Example 16

Evaluation of Anti-Cancer Activity)

Anti-cancer activity was evaluated using a method complying with an anti-cancer agent screening method (1990) using a human cancer cell panel which has been carried out by U.S. National Cancer Institute (NCI). Cells employed in experiments were from the following three cell lines: HL60 cell (cell line derived from human leukemia); HeLa cell (cell line derived from human uterine cancer); and HCT116 cell (cell line derived from human large intestine cancer). Media suitable for use of these cells are RPMI-1640 medium containing 10% FCS; a MEM medium containing 10% CS; and McCoy5A medium containing 10% FCS, respectively.

Cells suspended in themedium wereplated into 96-well plates at a concentration of 1×10⁵ cells/cm², followed by incubation overnight. Peptide solutions containing various concentrations of the peptide of the present invention were added to the plates, followed by incubation at 37° C. for 2 days in a CO₂ incubator. During culturing, the growth of the cells was measured by colorimetry using sulforhodamine B and the peptide concentration was categorized into any of GI₅₀ which is the concentration at which the growth is inhibited by 50% as compared to the control; TGI which is the concentration at which the number of cells is seemingly not increased or decreased from the time of addition of the peptide of the present invention, i.e., the growth is suppressed to the same number of cells as when the peptide was added: and LC₅₀ which is the concentration at which the number of cells is decreased to 50% of that at the time of addition of the peptide. Table 7 shows the LC₅₀ values of mast21R and KVL5.

TABLE 7
Evaluation of anti-cancer activity

| | mast21R | KLV5 |
|---|---|---|
| HCT116 cell | 2 × 10⁻⁶ | 1 × 10^{-4<} |
| Hela cell | 2 × 10⁻⁵ | 1 × 10^{-4<} |
| HL60 cell | 2 × 10⁻⁵ | 1 × 10^{-4<} |

When the LC₅₀ value was 1×10⁻⁴ or less, the peptide has anti-cancer activity. Therefore, Table 7 shows that mast21R has anti-cancer activity.

Thereafter, various cancer cells were employed to confirm the anti-cancer activity of mast21R. Each cancer cell was cultured in RPMI-1640 medium containing 10% FCS. The LC₅₀ values of mast21, mast21R, ALR6, and KVL6, obtained by the above-described method are shown in Table 8.

TABLE 8
Anticancer activity of peptides for various cancer
cells

| | | mast21 | mast21R | ALR6 | KVL6 |
|---|---|---|---|---|---|
| Bladdler | 5637 cell | 1 × 10⁻⁴ < | 5 × 10⁻⁵ | 1 × 10⁻⁴ < | 1 × 10⁻⁴ |
| cancer cell | EJ-1 cell | 7 × 10⁻⁵ | 2 × 10⁻⁵ | 1 × 10⁻⁴ < | 9 × 10⁻⁵ |
| Stomach | KATO III | 1 × 10⁻⁴ < | 5 × 10⁻⁵ | 1 × 10⁻⁴ < | 1 × 10⁻⁴ |
| cancer cell | cell | | | | |
| | SH-10-TC | 9 × 10⁻⁵ | 6 × 10⁻⁵ | 1 × 10⁻⁴ < | 6 × 10⁻⁵ |
| Breast | SR-BR-3 | 9 × 10⁻⁵ | 9 × 10⁻⁵ | 1 × 10⁻⁴ | 1 × 10⁻⁴ |
| cancer cell | MCF7 | 1 × 10⁻⁴ | 9 × 10⁻⁵ | 8 × 10⁻⁵ | 1 × 10⁻⁴ |
| Lung cancer | A549 | 7 × 10⁻⁵ | 5 × 10⁻⁵ | 1 × 10⁻⁴ < | 7 × 10⁻⁵ |
| cell | LK-2 | 1 × 10⁻⁴ < | 1 × 10⁻⁴ < | 1 × 10⁻⁴ < | 1 × 10⁻⁴ < |
| | EBC-1 | 1 × 10⁻⁴ | 7 × 10⁻⁵ | 1 × 10⁻⁴ < | 1 × 10⁻⁴ < |
| Prostate | PC-3 | 1 × 10⁻⁴ | 6 × 10⁻⁵ | 1 × 10⁻⁴ < | 1 × 10⁻⁴ < |
| cancer cell | DU-145 | 7 × 10⁻⁴ < | 9 × 10⁻⁵ | 1 × 10⁻⁴ < | 1 × 10⁻⁴ < |
| Glioblastoma | A172 | 1 × 10⁻⁴ < | 5 × 10⁻⁵ | 1 × 10⁻⁴ < | 1 × 10⁻⁴ < |
| cell | | | | | |
| Large | colo205 | 1 × 10⁻⁴ < | 5 × 10⁻⁵ | 1 × 10⁻⁴ < | 1 × 10⁻⁴ |
| insetine | HCT-15 | 8 × 10⁻⁵ | 3 × 10⁻⁵ | 1 × 10⁻⁴ < | 8 × 10⁻⁵ |
| cancer cell | | | | | |
| Uterine | D98-AH2 | 1 × 10⁻⁴ < | 5 × 10⁻⁵ | 1 × 10⁻⁴ < | 1 × 10⁻⁴ < |
| cancer cell | | | | | |
| Ovarian | NIH | 1 × 10⁻⁴ | 8 × 10⁻⁵ | 1 × 10⁻⁴ < | 1 × 10⁻⁴ < |
| cancer cell | OVCAR-3 | | | | |
| Kidney | ACHN | 8 × 10⁻⁵ | 3 × 10⁻⁵ | 1 × 10⁻⁴ < | 8 × 10⁻⁵ |
| cancer cell | | | | | |

Mast21R exhibited anti-cancer activity against various cancer cells. Mast21 was also found to have anti-cancer activity against some cancer cell(s), though the anti-cancer activity of mast21R was two times or more greater than that of mast21.

ALR6 and KVL6 also exhibited anti-cancer activity against some cancer cell(s).

### Example 17

Evaluation of Antibacterial Activity Against Plant Pathogenic Bacteria

Mast21, mast21R, ALR6, and KVL6 were evaluated with respect to antibacterial activity against Erwinia carotovora (NBRC3380) which is a plant pathogenic bacterium causing soft rot. Specifically, a minimum concentration of a peptide capable of inhibiting the growth of bacteria was determined using microtiter plates. Bacteria were cultured for 16 h in culture medium (Difco Nutrient Broth 8 g, NaCl 5 g/L, pH 7.0), followed by measurement of absorbance at A₆₀₀. Each bacterial strain was diluted into a final concentration of 1×10⁶ CFU/ml. For each peptide, 5 mM aqueous solution was prepared, and thereafter, was subjected to serial dilution. The peptide serial dilutions (50 μl for each) were added to respective wells of the microplate to which 50 μl of the bacterial solution had been added (final cell concentration: 5×10⁵ CFU/ml, final peptide concentration: 0 to 50 μM). The plate was allowed to stand at 30° C. for 16 to 24 h for culture so that a minimum concentration which inhibits the bacterial growth (minimum inhibitory concentration: MIC) was determined (μM and μg/ml). The result is shown in Table 9. The unit of concentration described in Table 9 is μM.

TABLE 9
Antibacterial activity of peptides for
Erwinia carotovora (NBRC3380)

| Name of | | | MIC |
|---|---|---|---|
| peptide | Amino acid sequence | MIC(μM) | (μg/ml) |
| mast 21 | KNWKGIAGMAKKLLGKNWKLM | 3.1 | 3.7 |
| mast 21R | RNWRGIAGMARRLLGRNWRLM | 1.6 | 2 |
| ALR6 | ALRALRALRALRALRALR | 3.1 | 3.2 |
| KVL6 | KVLKVLKVLKVLKVLKVL | 3.1 | 3.2 |

All of mast21, mast21R, ALR6, and KVL6 exhibited a high level of antibacterial activity against Erwinia carotovora (NBRC3380) which is a plant pathogenic bacterium causing soft rot. Particularly, mast21R exhibited antibacterial activity about two times greater than mast21.

### Example 18

Evaluation of Suppression of Apoptosis

HL60 cells which had been treated with a potent apoptosis inducing reagent, cycloheximide, were employed as apoptotic cells. Usually, when apoptosis is induced in a cell, aggregation of chromatins and fragmentation of chromosomes occurs in the cell nucleus. Thereafter, within several hours, a poptosis bodies were formed. When apoptotic cells treated with cycloheximide were observed using a phase-contrast microscope, apoptosis bodies were confirmed in 80% or more of all of the cells.

The HL60 cells were suspended in RPMI-1640 medium containing 10% FCS to a concentration of 2×10⁵ cells/ml. A peptide solution containing cycloheximide having a final concentration of 1×10⁷ M and fluorescent labeled mast21R was added to the cell culture. The cell culture was cultured at 37° C. for several hours in a CO₂ incubator, followed by observation using a fluorescence microscope. As a result, it was confirmed that fluorescent labeled mast21R bound to the apoptotic cells.

Thus, it was confirmed that mast21R recognizes and binds to apoptotic cells. Therefore, the effect of mast21R suppressing the induction of apoptosis was evaluated using a phase-contrast microscope.

Cells were suspended in RPMI-1640 medium containing 10% FCS to a concentration of 2×10⁵ cells/ml. Cycloheximide was added to the suspension to a final concentration of 1×10⁻⁷ M. These cells were cultured at 37° C. for several hours in a CO₂ incubator. Thereafter, peptide solutions containing various concentrations of mast21R were added to the apoptotic cell culture, and a change in the cells was observed over time. In controls without mast21R, apoptosis bodies were generated within several hours after cycloheximide treatment. In contrast, in the solution containing mast21R, no formation of an apoptosis body was found in all cells 12 hours after cycloheximide treatment (data not shown).

All patents, patent applications, journal articles and other references discussed or mentioned herein are incorporated by reference in their entireties.

Various other modifications will be apparent to and can be readily made by those skilled in the art without departing from the scope and spirit of this invention. Accordingly, it is not intended that the scope of the claims appended hereto be limited to the description as set forth herein, but rather that the claims be broadly construed.

What is claimed is:

1. A peptide consisting of amino acid sequence comprising an amino acid sequence Z¹X¹X²X³Z²X⁴X⁵Z³X⁶X⁷X⁸Z⁴X⁹, wherein X¹-X⁹ are any amino acids and at least two amino acids of Z¹-Z⁴ are basic amino acids, or an analog thereof.

2. The peptide of claim 1 or an analog thereof, wherein the basic amino acids are lysine (K) or arginine (R).

3. The peptide of claim 1 or an analog thereof, wherein at least two amino acids of the X¹-X⁹ are hydrophobic amino acids.

4. The peptide of claim 1 comprising a sequence selected from the group consisting of ALR, WALR, WGALR, RLAWG, GWALR, RVL, KVL, RVG, KVG, GVR, VGR, RVA, RSV, RVS, KVS, SVK, and VSK, or an analog thereof.

5. The peptide of claim 1 comprising a sequence selected from the group consisting of ALR, RLAWG, GWALR, RVL, KVL, RVG, and KVG, or an anolog thereof.

6. The peptide of claim 1 comprising a sequence selected from the group consisting of ALR, KVL, and RVG, or an anolog thereof.

7. A peptide capable of specifically acting on a membrane of a microorganism, comprising a sequence selected from the group consisting of ALR, WALR, WGALR, RLAWG, GWALR, RVL, KVL, RVG, KVG, GVR, VGR, RVA, RSV, RVS, KVS, SVK, and VSK, or an anolog thereof.

8. The peptide of claim 7 or an analog thereof, wherein the microorganism causes putrefaction of food or industrial products.

9. The peptide of claim 7 comprising a sequence selected from the group consisting of ALR, RLAWG, GWALR, RVL, KVL, RVG, and KVG, or an analog thereof.

10. The peptide of claim 7 comprising a sequence selected from the group consisting of ALR, KVL, and RVG, or an analog thereof.

11. A peptide capable of specifically acting on a membrane of an animal cell having an abnormality, comprising a sequence selected from the group consisting of ALR, WALR, WGALR, RLAWG, GWALR, RVL, KVL, RVG, KVG, GVR, VGR, RVA, RSV, RVS, KVS, SVK, and VSK, or an analog thereof.

12. The peptide of claim 11 or an analog thereof, wherein the animal cell having an abnormality is a cancer cell.

13. The peptide of claim 12, comprising a sequence selected from the group consisting of ALR, RLAWG, GWALR, RVL, KVL, RVG, and KVG, or an analog thereof.

14. The peptide of claim 12, comprising a sequence selected from the group consisting of ALR, KVL, and RVG, or an analog thereof.

15. The peptide of claim 11 or an analog thereof, wherein the animal cell having an abnormality is an apoptotic cell.

16. The peptide of claim 15, comprising a sequence selected from the group consisting of ALR, RLAWG, GWALR, RVL, KVL, RVG, and KVG, or an analog thereof.

17. The peptide of claim 15, comprising a sequence selected from the group consisting of ALR, KVL, and RVG, or an analog thereof.

18. A peptide, comprising:
i) a first sequence selected from the group consisting of ALR, WALR, WGALR, RLAWG, GWALR, RVL, KVL, RVG, KVG, GVR, VGR, RVA, RSV, RVS, KVS, SVK, and VSK;
ii) a second sequence selected from the group consisting of ALR, WALR, WGALR, RLAWG, GWALR, RVL, KVL, RVG, KVG, GVR, VGR, RVA, RSV, RVS, KVS, SVK, and VSK; and
iii) a third sequence selected from the group consisting of ALR, WALR, WGALR, RLAWG, GWALR, RVL, KVL, RVG, KVG, GVR, VGR, RVA, RSV, RVS, KVS, SVK, and VSK.

19. A peptide, comprising a repeat of at least three sequences of KVL or ALR.

20. A peptide comprising an amino acid sequence selected from the group consisting of SEQ ID NO: 11 to SEQ ID NO: 122, or an analog thereof.

21. A peptide capable of specifically acting on a membrane of a microorganism, comprising an amino acid sequence selected from the group consisting of SEQ ID NO: 11 to SEQ ID NO: 122, or an analog thereof.

22. The peptide of claim 21 or an analog thereof, wherein the microorganism causes putrefaction of food or industrial products.

23. A peptide capable of specifically acting on a membrane of an animal cell having an abnormality, comprising an amino acid sequence selected from the group consisting of SEQ ID NO: 11 to SEQ ID NO: 122, or an analog thereof.

24. The peptide of claim 23 or an analog thereof, wherein the animal cell having an abnormality is a cancer cell.

25. The peptide of claim 23 or an analog thereof, wherein the animal cell having an abnormality is an apoptotic cell.

26. A peptide comprising an amino acid sequence selected from the group consisting of SEQ ID NO: 11 to SEQ ID NO: 122 or a variant thereof, wherein the variant has at least one amino acid deletion, addition, and/or substitution in the amino acid sequence, and maintains a property of specifically acting on a membrane of a microorganism.

27. The peptide of claim 26 or a variant thereof, wherein the microorganism causes putrefaction of food or industrial products.

28. A peptide comprising an amino acid sequence selected from the group consisting of SEQ ID NO: 11 to SEQ ID NO: 122 or a variant thereof, wherein the variant has at least one amino acid deletion, addition, and/or substitution in the amino acid sequence, and maintains a property of specifically acting on a membrane of an animal cell having an abnormality.

29. The peptide of claim 28 or a variant thereof, wherein the animal cell having an abnormality is a cancer cell.

30. The peptide of claim 28 or a variant thereof, wherein the animal cell having an abnormality is an apoptotic cell.

31. A peptide comprising an amino acid sequence selected from the group consisting of SEQ ID NO: 11 to SEQ ID NO: 122 or a variant thereof, wherein the variant has at least one conservative amino acid substitution in the amino acid sequence, and maintains a property of specifically acting on a membrane of a microorganism.

32. The peptide of claim 31 or a variant thereof, wherein the microorganism causes putrefaction of food or industrial products.

33. A peptide comprising an amino acid sequence selected from the group consisting of SEQ ID NO: 11 to SEQ ID NO: 122 or a variant thereof, wherein the variant has at least one conservative amino acid substitution in the amino acid sequence, and maintains a property of specifically acting on a membrane of an animal cell having an abnormality.

34. The peptide of claim 33 or a variant thereof, wherein the animal cell having an abnormality is a cancer cell.

35. The peptide of claim 33 or a variant thereof, wherein the animal cell having an abnormality is an apoptotic cell.

36. A peptide comprising a sequence RNWRGIAGMARRLLGRNWRLM or an analog thereof, wherein the peptide or the analog thereof has an ability to act on a membrane of a microorganism at least two fold higher than a peptide comprising a sequence KNWRGIAGMAKKLLGKNWKLM.

37. The peptide of claim 36 or an analog thereof, wherein the microorganism causes putrefaction of food or industrial products.

38. A peptide comprising a sequence RNWRGIAGMARRLLGRNWRLM or an analog thereof, wherein the peptide or the analog thereof has an ability to act on a membrane of an animal cell having an abnormality at least two fold higher than a peptide comprising a sequence KNWRGIAGMAKKLLGKNWKLM.

39. The peptide of claim 38 or an analog thereof, wherein the animal cell having an abnormality is a cancer cell.

40. The peptide of claim 38 or an analog thereof, wherein the animal cell having an abnormality is an apoptotic cell.

41. A peptide comprising a sequence RNWRGIAGMARRLLGRNWRLM or a variant thereof, wherein the variant has at least one amino acid deletion, addition, and/or substitution in the sequence-, maintains a property of specifically acting on a membrane of a microorganism, and has an ability to act on a membrane of a microorganism at least two fold higher than a peptide having a sequence KNWRGIAGMAKKLLGKNWKLM.

42. The peptide of claim 41 or a variant thereof, wherein the microorganism causes putrefaction of food or industrial products.

43. A peptide comprising a sequence RNWRGIAGMARRLLGRNWRLM or a variant thereof, wherein the variant has at least one amino acid deletion, addition, and/or substitution in the sequence, maintains a property of specifically acting on a membrane of an animal cell having an abnormality, and has an ability to act on a membrane of an animal cell having an abnormality at least two fold higher than a peptide having a sequence KNWRGIAGMAKKLLGKNWKLM.

44. The peptide of claim 43 or an analog thereof, wherein the animal cell having an abnormality is a cancer cell.

45. The peptide of claim 43 or an analog thereof, wherein the animal cell having an abnormality is an apoptotic cell.

46. A peptide comprising a sequence RNWRGIAGMARRLLGRNWRLM or a variant thereof, wherein the variant has at least one conservative amino acid substitution excluding K or R in the sequence, maintains a property of specifically acting a membrane of a microorganism, and has an ability to act on a membrane of a microorganism at least two fold higher than a peptide having a sequence KNWRGIAGMAKKLLGKNWKLM.

47. The peptide of claim 46 or an analog thereof, wherein the microorganism causes putrefaction of food or industrial products.

48. A peptide comprising a sequence RNWRGIAGMARRLLGRNWRLM or a variant thereof, wherein the variant has at least one conservative substitution excluding K or R in the sequence, maintains a property of specifically acting on a membrane of an animal cell having an abnormality, and has an ability to act on an ability to act on a membrane of an animal cell having an abnormality at least two fold higher than a peptide having a sequence KNWRGIAGMAKKLLGKNWKLM.

49. The peptide of claim 48 or an analog thereof, wherein the animal cell having an abnormality is a cancer cell.

50. The peptide of claim 48 or an analog thereof, wherein the animal cell having an abnormality is an apoptotic cell.

51. A library, comprising a plurality of nucleic acid sequences, each nucleic acid sequence comprising:
(1) a first cassette comprising a base sequence encoding a first peptide;
(2) a second cassette comprising a base sequence encoding a second peptide, said base sequence having the same reading frame as that of the base sequence encoding the first peptide, wherein the second peptide comprises a site allowing flexible movement of the first peptide: and
(3) a third cassette comprising a base sequence essentially required for transcription and translation of the first and the second cassette, the third cassette being operatively linked to the first and second cassettes,
wherein the number of the nucleic acid sequences in the library whose first cassettes are different from one another is at least two.

52. The library of claim 51, wherein the second cassette further comprises a base sequence encoding a tag sequence.

53. The library of claim 51, wherein the first cassette does not-comprise a termination codon.

54. The library of claim 51, wherein the number of the nucleic acid sequences whose first cassettes are different from one another is at least 100.

55. The library of claim 51, wherein the number of the nucleic acid sequences whose first cassettes are different from one another is at least 1000.

56. A vector comprising the library of claim 51.

57. A method for screening for a nucleic acid encoding a peptide capable of acting on a biological membrane, the method comprising the steps of.:
constructing a DNA library;
preparing peptides by transcription and translation of DNAs of the library in a cell-free system, and forming complexes of the peptide, a ribosome, and mRNA; and
selecting the complex capable of specifically binding to a membrane model.

58. The method of claim 57, wherein the DNA library comprises the library of claim 51.

59. The method of claim 57, wherein the DNA library comprises the library of claim 53.

60. The method of claim 57, wherein the membrane model is an artificial lipid bilayer imitating a cell membrane structure of an organism.

61. The method of claim 57, wherein the membrane model is a membrane model immobilized on a solid phase.

62. The method of claim 61, wherein the solid phase is a magnetic bead.

63. The method of claim 57, further comprising reverse-transcribing mRNA in the selected complex to DNA.

64. The method of claim 63, wherein a DNA library is prepared using DNA obtained in the reverse-transcription step, and the complex forming step, the complex selecting step, and the reverse-transcription step are repeated.

65. The method of claim 64, wherein the number of the repetitions is at least 4.

66. A pharmaceutical composition for killing a microorganism, comprising the peptide of any one of claims 1 to 10, 18 to 21, 26, 31, 36, 41, and 46, or an analog or variant thereof.

67. A pharmaceutical composition for preventing putrefaction of food or industrial products, comprising the peptide of any one of claims 1 to 10, 18 to 21, 26, 31, 36, 41, and 46, or an analog or variant thereof.

68. A pharmaceutical composition for killing a microorganism, comprising the peptide of any one of claims 6, 10, 19, 21, 26, 31, 36, 41, and 46, or an analog or variant thereof.

69. A pharmaceutical composition for preventing putrefaction of food or industrial products, comprising the peptide of any one of claims 6, 10, 19, 21, 26, 31, 36, 41, and 46, or an analog or variant thereof.

70. A pharmaceutical composition for treating an infectious disease caused by a microorganism, comprising the peptide of any one of claims 1 to 10, 18 to 21, 26, 31, 36, 41, and 46, or an analog or variant thereof.

71. A pharmaceutical composition for treating an infectious disease caused by a microorganism, comprising the peptide of any one of claims 6, 10, 19, 21, 26, 31, 36, 41, and 46, or an analog or variant thereof.

72. An antibiotic comprising the peptide of any one of claims 1 to 10, 18 to 21, 26, 31, 36, 41, and 46, or an analog or variant thereof.

73. A pharmaceutical delivery substance for delivering a drug to a site infected with a microorganism, comprising the peptide of any one of claims 1 to 10, 18 to 21, 26, 31, 36, 41, and 46, or an analog or variant thereof.

74. A pharmaceutical delivery substance for delivering a drug to a site infected with a microorganism, comprising the peptide of any one of claims 6, 10, 19, 21, 26, 31, 36, 41, and 46, or an analog or variant thereof.

75. A pharmaceutical composition for treating a cancer, comprising the peptide of any one of claims 1 to 6, 12 to 14, 18 to 20, 24, 29, 34, 39, 44, and 49, or an analog or variant thereof.

76. A pharmaceutical composition for treating a cancer, comprising the peptide of any one of claims 6, 14, 19, 24, 29, 34, 39, 44, and 49, or an analog or variant thereof.

77. A pharmaceutical delivery substance for delivering a drug to a cancer lesion site, comprising the peptide of any one of claims 1 to 6, 12 to 14, 18 to 20, 24, 29, 34, 39, 44, and 49, or an analog or variant thereof.

78. A pharmaceutical delivery substance for delivering a drug to a cancer lesion site, comprising the peptide of any one of claims 6, 14, 19, 24, 29, 34, 39, 44, and 49, or an analog or variant thereof.

79. A pharmaceutical composition for suppressing apoptosis, comprising the peptide of any one of claims 1 to 6, 15 to 20, 25, 30, 35, 40, 45, and 50, or an analog or variant thereof.

80. A pharmaceutical composition for suppressing apoptosis, comprising the peptide of any one of claims 6, 17, 19, 25, 30, 35, 40, 45, and 50, or an analog or variant thereof.

81. A pharmaceutical delivery substance for delivering a drug to a site undergoing apoptosis, comprising the peptide of any one of claims 1 to 6, 15 to 20, 25, 30, 35, 40, 45, and 50, or an analog or variant thereof.

82. A pharmaceutical delivery substance for delivering a drug to a site undergoing apoptosis, comprising the peptide of any one of claims 6, 17, 19, 25, 30, 35, 40, 45, and 50, or an analog or variant thereof.

83. A kit for screening for a nucleic acid encoding a peptide capable of acting on a biological membrane, the kit comprising:
a lipid for preparing a membrane model.

84. The kit of claim 83, further comprising an enzyme and ribosome for forming a peptide-ribosome-mRNA complex in a cell-free system.

85. The kit of claim 84, wherein the enzyme and the ribosome are provided as cell free extracts.

86. The kit of claim 85, wherein the cell free extract is S30 extract, i.e., E. coli extract.

87. The kit of claim 83, further comprising the library of claim 51.

88. The kit of claim 83, further comprising the library of claim 53.

89. A pharmaceutical composition for killing a microorganism, comprising a peptide having an amino acid sequence selected from the group consisting of SEQ ID NOs: 5, 107, 121, and 122, or an analog or variant thereof.

90. A pharmaceutical composition for preventing putrefaction food or industrial products, comprising a peptide having an amino acid sequence selected from the group consisting of SEQ ID NOs: 5, 107, 121, and 122, or an analog or variant thereof.

91. A pharmaceutical composition for treating an infectious disease caused by a microorganism, comprising a peptide having an amino acid sequence selected from the group consisting of SEQ ID NOs: 5, 107, 121, and 122, or an analog or variant thereof.

92. The pharmaceutical composition of claim 91, wherein the microorganism is pathogenic to an animal.

93. The pharmaceutical composition of claim 91, wherein the microorganism is pathogenic to a plant.

94. The pharmaceutical composition of claim 93, wherein the plant pathogenic microorganism is Erwinia carotovora.

95. A pharmaceutical composition for treating a cancer, comprising a peptide having an amino acid sequence selected from the group consisting of SEQ ID NOs: 5, 107, 121, and 122, or an analog or variant thereof.

96. The pharmaceutical composition of claim 95, wherein the cancer is selected from the group consisting of bladder cancer, stomach cancer, breast cancer, lung cancer, prostate cancer, glioblastoma, large intestine cancer, uterine cancer, ovarian cancer, kidney cancer, and leukemia.


SEQUENCE LISTING (122 sequences)

SEQ ID NO: 1 - DNA, 17 bp, artificial
  T7 promotor sequence
taatacgactcactata

SEQ ID NO: 2 - DNA, 6 bp, artificial
  Shine-Dalgano seuqence
aaggag

SEQ ID NO: 3 - DNA, 21 bp, artificial
  5′ stem-loop sequence
gggagaccacaacggtttccc

SEQ ID NO: 4 - protein, 4 aa, artificial
  synthetic peptide
GGGS

SEQ ID NO: 5 - protein, 21 aa, Artificial
  synthetic peptide
KNWKGIAGMAKKLLGKNWKLM

SEQ ID NO: 6 - DNA, 21 bp, artificial
  primer
tgctctagagcagcctccaga

SEQ ID NO: 7 - DNA, 21 bp, artificial
  primer
cggaattcccgcacaccagta

SEQ ID NO: 8 - DNA, 21 bp, artificial
  primer
gatctcgatcccgcgaaatta

SEQ ID NO: 9 - DNA, 39 bp, artificial
  primer
gcgaaattaatacgactcactatagggagaccacaacgg

SEQ ID NO: 10 - protein, 14 aa, mastoparan
INWKGIAAMAKKLL

SEQ ID NO: 11 - protein, 21 aa, Artificial
  peptide coded by random synthetic DNA library
VWAWVFGASTRERARVGWQGY

SEQ ID NO: 12 - protein, 21 aa, Artificial
  synthetic peptide coded by random DNA library
CFVLPGVRPCSSHILTLSFSY

SEQ ID NO: 13 - protein, 21 aa, Artificial
  synthetic peptide coded by random DNA library
EGVRDMFRRCLWISLRSWCVH

SEQ ID NO: 14 - protein, 21 aa, Artificial
  synthetic peptide coded by random DNA library
GGVYASCASYLLALLSRVGGN

SEQ ID NO: 15 - protein, 19 aa, Artificial
  synthetic peptide coded by random DNA library
SDSASVSRVGGLWPTCCPH

SEQ ID NO: 16 - protein, 21 aa, Artificial
  synthetic peptide coded by random DNA library
WGRVDNSGSWGRVGAPWRYLH

SEQ ID NO: 17 - protein, 21 aa, Artificial
  synthetic peptide coded by random DNA library
CAAVYLVTSLFGIVTGVREDH

SEQ ID NO: 18 - protein, 21 aa, Artificial
  synthetic peptide coded by random DNA library
DSRGVRAFACDYVLFVLWVPY

SEQ ID NO: 19 - protein, 21 aa, Artificial
  synthetic peptide coded by random DNA library
DLAPVRVGFYNAYRELRVFRY

SEQ ID NO: 20 - protein, 21 aa, Artificial
  synthetic peptide coded by random DNA library
RVGALYYFMVWYLMWFFLLFH

SEQ ID NO: 21 - protein, 20 aa, Artificial
  synthetic peptide coded by random DNA library
PVLDAGSVYLGYLGVRFLSY

SEQ ID NO: 22 - protein, 21 aa, Artificial
  synthetic peptide coded by random DNA library
VRNRVIARVGGVPYVGGPCYN

SEQ ID NO: 23 - protein, 21 aa, Artificial
  synthetic peptide coded by random DNA library
ACCLVRFYSHGRGKRVGFLWY

SEQ ID NO: 24 - protein, 21 aa, Artificial
  synthetic peptide coded by random DNA library
LRYSGLLGFPLWVGRIFVCVD

SEQ ID NO: 25 - protein, 21 aa, Artificial
  synthetic peptide coded by random DNA library
RMRCVSLELVVYGGGVRMWEN

SEQ ID NO: 26 - protein, 21 aa, Artificial
  synthetic peptide coded by random DNA library
FMGYGRSVWVVSSSLVLCIYD

SEQ ID NO: 27 - protein, 21 aa, Artificial
  synthetic peptide coded by random DNA library
ARMLWGRGTTLLLIRRRVSAY

SEQ ID NO: 28 - protein, 20 aa, Artificial
  synthetic peptide coded by random DNA library
VDLSWYASCRVSICVFVVVY

SEQ ID NO: 29 - protein, 21 aa, Artificial
  synthetic peptide coded by random DNA library
WPNYQSREHMRVSSRMYYYFY

SEQ ID NO: 30 - protein, 21 aa, Artificial
  synthetic peptide coded by random DNA library
CVVRVSNVKAAALIPGVVSRH

SEQ ID NO: 31 - protein, 21 aa, Artificial
  synthetic peptide coded by random DNA library
WWCLLGYWALGGNHSAKVSSY

SEQ ID NO: 32 - protein, 20 aa, Artificial
  synthetic peptide coded by random DNA library
YGSYLEALWWGTSACWALRY

SEQ ID NO: 33 - protein, 21 aa, Artificial
  synthetic peptide coded by random DNA library
GVAVDCAVVGWALRVLGVHSY

SEQ ID NO: 34 - protein, 21 aa, Artificial
  synthetic peptide coded by random DNA library
RCLEAGKIWWGALRSHLAVYD

SEQ ID NO: 35 - protein, 21 aa, Artificial
  synthetic peptide coded by random DNA library
GSGSAVGWALRSYASGLAIAY

SEQ ID NO: 36 - protein, 21 aa, Artificial
  synthetic peptide coded by random DNA library
HAWARWMGWGHGGVLSWALRY

SEQ ID NO: 37 - protein, 21 aa, Artificial
  synthetic peptide coded by random DNA library
FVSWALRYSRCLVWLCWFPNY

SEQ ID NO: 38 - protein, 21 aa, Artificial
  synthetic peptide coded by random DNA library
VKGNPVFDHRHFSLWGALREY

SEQ ID NO: 39 - protein, 21 aa, Artificial
  synthetic peptide coded by random DNA library
FVQHWSFTAGSRSDRAPYPGH

SEQ ID NO: 40 - protein, 21 aa, Artificial
  synthetic peptide coded by random DNA library
AGWVNARRMWSLMPLMWLWSY

SEQ ID NO: 41 - protein, 21 aa, Artificial
  synthetic peptide coded by random DNA library
DRTTGRWFYIRRTAEVLGWTY

SEQ ID NO: 42 - protein, 21 aa, Artificial
  synthetic peptide coded by random DNA library
RFINPTSHCFGSLSLWRQLSY

SEQ ID NO: 43 - protein, 21 aa, Artificial
  synthetic peptide coded by random DNA library
VGCLVSVGSVWGCSSVVVRVY

SEQ ID NO: 44 - protein, 21 aa, Artificial
  synthetic peptide coded by random DNA library
RMESGAPLAAYGKMRLRPGTH

SEQ ID NO: 45 - protein, 21 aa, Artificial
  synthetic peptide coded by random DNA library
VWNRVIARCGGVLYVGGPCTN

SEQ ID NO: 46 - protein, 21 aa, Artificial
  synthetic peptide coded by random DNA library
SGHMHSYWPTTWILVLIRRTY

SEQ ID NO: 47 - protein, 21 aa, Artificial
  synthetic peptide coded by random DNA library
LEVLVWYSLWSYWLDVAAASH

SEQ ID NO: 48 - protein, 20 aa, Artificial
  synthetic peptide coded by random DNA library
SWGGGFYDWSYVGGGAYWAY

SEQ ID NO: 49 - protein, 20 aa, Artificial
  synthetic peptide coded by random DNA library
MGLFRSYKYRFVHDSESSFN

SEQ ID NO: 50 - protein, 21 aa, Artificial
  synthetic peptide coded by random DNA library
MALYLAWYGCSDSAVVMLADD

SEQ ID NO: 51 - protein, 21 aa, Artificial
  synthetic peptide coded by random DNA library
MYCWRMLANSCAARMVLAMRN

SEQ ID NO: 52 - protein, 21 aa, Artificial
  synthetic peptide coded by random DNA library
VIVNVAVLYRRCWPCAEFWPY

SEQ ID NO: 53 - protein, 21 aa, Artificial
  synthetic peptide coded by random DNA library
RLGSFYPLLWRLVSHEYSLWH

SEQ ID NO: 54 - protein, 21 aa, Artificial
  synthetic peptide coded by random DNA library
RYWFGRWRCFYGPFVSSYFLY

SEQ ID NO: 55 - protein, 21 aa, Artificial
  synthetic peptide coded by random DNA library
VCCCRCLPWSYMCEWGSMRLY

SEQ ID NO: 56 - protein, 21 aa, Artificial
  synthetic peptide coded by random DNA library
VLKIHSWHNWVYGVMLYDMEY

SEQ ID NO: 57 - protein, 21 aa, Artificial
  synthetic peptide coded by random DNA library
MGYAWDLGLRMGPYFLMDLIN

SEQ ID NO: 58 - protein, 20 aa, Artificial
  synthetic peptide coded by random DNA library
SDKCAPVCYVMDRLCLANWD

SEQ ID NO: 59 - protein, 14 aa, Artificial
  synthetic peptide coded by random DNA library
RPVFRTYRSVVKSG

SEQ ID NO: 60 - protein, 14 aa, Artificial
  synthetic peptide coded by random DNA library
NRACLKRPRYLRKH

SEQ ID NO: 61 - protein, 14 aa, Artificial
  synthetic peptide coded by random DNA library
KACVRFSKSTSKRY

SEQ ID NO: 62 - protein, 14 aa, Artificial
  synthetic peptide coded by random DNA library
RFRPKAVRYRIKFN

SEQ ID NO: 63 - protein, 14 aa, Artificial
  synthetic peptide coded by random DNA library
RSHFRRVKRHSKTP

SEQ ID NO: 64 - protein, 14 aa, Artificial
  synthetic peptide coded by random DNA library
KDVLRNHKHSDRVG

SEQ ID NO: 65 - protein, 14 aa, Artificial
  synthetic peptide coded by random DNA library
KSARKVLKLYRKIT

SEQ ID NO: 66 - protein, 14 aa, Artificial
  synthetic peptide coded by random DNA library
KKNSCRDGRFSRKC

SEQ ID NO: 67 - protein, 14 aa, Artificial
  synthetic peptide coded by random DNA library
KGYFRGRRSYLRAF

SEQ ID NO: 68 - protein, 14 aa, Artificial
  synthetic peptide coded by random DNA library
KGCAKVLKRITRHI

SEQ ID NO: 69 - protein, 14 aa, Artificial
  synthetic peptide coded by random DNA library
KGRHRHCRYILRGN

SEQ ID NO: 70 - protein, 14 aa, Artificial
  synthetic peptide coded by random DNA library
KCTFRRRRLIIKPS

SEQ ID NO: 71 - protein, 14 aa, Artificial
  synthetic peptide coded by random DNA library
KTDWFRVLMTFLMD

SEQ ID NO: 72 - protein, 14 aa, Artificial
  synthetic peptide coded by random DNA library
RGFVRLIKPYAEAS

SEQ ID NO: 73 - protein, 13 aa, Artificial
  synthetic peptide coded by random DNA library
RNLCRSLRSHLEA

SEQ ID NO: 74 - protein, 14 aa, Artificial
  synthetic peptide coded by random DNA library
KIPGRFTRAGRKTT

SEQ ID NO: 75 - protein, 14 aa, Artificial
  synthetic peptide coded by random DNA library
KSDHRVAKNLPKTI

SEQ ID NO: 76 - protein, 14 aa, Artificial
  synthetic peptide coded by random DNA library
RRTGRIDKVSVKAY

SEQ ID NO: 77 - protein, 14 aa, Artificial
  synthetic peptide coded by random DNA library
KVLIKLAKCCIRIS

SEQ ID NO: 78 - protein, 14 aa, Artificial
  synthetic peptide coded by random DNA library
RAACRDSKLCSRYY

SEQ ID NO: 79 - protein, 14 aa, Artificial
  synthetic peptide coded by random DNA library
KHFVRCPKCAVRSS

SEQ ID NO: 80 - protein, 14 aa, Artificial
  synthetic peptide coded by random DNA library
KISDRNSKHHCRSS

SEQ ID NO: 81 - protein, 14 aa, Artificial
  synthetic peptide coded by random DNA library
KVGLIVDKASVKTA

SEQ ID NO: 82 - protein, 14 aa, Artificial
  synthetic peptide coded by random DNA library
RDVCKSSRHSHKGS

SEQ ID NO: 83 - protein, 14 aa, Artificial
  synthetic peptide coded by random DNA library
RFVSKGTDAINRRS

SEQ ID NO: 84 - protein, 14 aa, Artificial
  synthetic peptide coded by random DNA library
KGNCRLYRLRCKVV

SEQ ID NO: 85 - protein, 14 aa, Artificial
  synthetic peptide coded by random DNA library
RLLLKAVRFCCKCF

SEQ ID NO: 86 - protein, 13 aa, Artificial
  synthetic peptide coded by random DNA library
KGGGKVGKHTRSR

SEQ ID NO: 87 - protein, 14 aa, Artificial
  synthetic peptide coded by random DNA library
RHFRKNCKFCHRHC

SEQ ID NO: 88 - protein, 14 aa, Artificial
  synthetic peptide coded by random DNA library
KRCTKVYRAYTKLT

SEQ ID NO: 89 - protein, 14 aa, Artificial
  synthetic peptide coded by random DNA library
KSYGKAPKFVGRIC

SEQ ID NO: 90 - protein, 14 aa, Artificial
  synthetic peptide coded by random DNA library
RAAIRHFRSATKRP

SEQ ID NO: 91 - protein, 14 aa, Artificial
  synthetic peptide coded by random DNA library
KYSARFCKYGGRSH

SEQ ID NO: 92 - protein, 14 aa, Artificial
  synthetic peptide coded by random DNA library
RFTARVRKSVFRSC

SEQ ID NO: 93 - protein, 14 aa, Artificial
  synthetic peptide coded by random DNA library
KVYSRSSKSAHKCF

SEQ ID NO: 94 - protein, 14 aa, Artificial
  synthetic peptide coded by random DNA library
KRAYKDARHIYLCS

SEQ ID NO: 95 - protein, 14 aa, Artificial
  synthetic peptide coded by random DNA library
KIFVRTIRAAHKRD

SEQ ID NO: 96 - protein, 13 aa, Artificial
  synthetic peptide coded by random DNA library
KGGGKVGKHTRSR

SEQ ID NO: 97 - protein, 14 aa, Artificial
  synthetic peptide coded by random DNA library
KSLTKCCKVLRLSC

SEQ ID NO: 98 - protein, 14 aa, Artificial
  synthetic peptide coded by random DNA library
RCDIKSVKHILRCS

SEQ ID NO: 99 - protein, 14 aa, Artificial
  synthetic peptide coded by random DNA library
KASVRNSKNLPRFC

SEQ ID NO: 100 - protein, 14 aa, Artificial
  synthetic peptide coded by random DNA library
KGAFRLAKHLIRHY

SEQ ID NO: 101 - protein, 14 aa, Artificial
  synthetic peptide coded by random DNA library
RHVPKANKGADRSC

SEQ ID NO: 102 - protein, 14 aa, Artificial
  synthetic peptide coded by random DNA library
KTSWVRAAALVVVH

SEQ ID NO: 103 - protein, 13 aa, Artificial
  synthetic peptide coded by random DNA library
KSVNKDVRISLRD

SEQ ID NO: 104 - protein, 14 aa, Artificial
  synthetic peptide coded by random DNA library
KCIARRGRLPVKRY

SEQ ID NO: 105 - protein, 14 aa, Artificial
  synthetic peptide coded by random DNA library
KVLFRHARSSCKHY

SEQ ID NO: 106 - protein, 21 aa, Artificial
  synthetic peptide
KNRKGIAGMAKKLLGNKWKLM

SEQ ID NO: 107 - protein, 21 aa, Artificial
  synthetic peptide
RNWRGIAGMARRLLGRNWRLM

SEQ ID NO: 108 - protein, 5 aa, Artificial
  synthetic peptide
RLAWG

SEQ ID NO: 109 - protein, 5 aa, Artificial
  synthetic peptide
GWALR

SEQ ID NO: 110 - protein, 3 aa, Artificial
  synthetic peptide
RVL

SEQ ID NO: 111 - protein, 3 aa, Artificial
  synthetic peptide
RVG

SEQ ID NO: 112 - protein, 3 aa, Artificial
  synthetic peptide
KVG

SEQ ID NO: 113 - protein, 3 aa, Artificial
  synthetic peptide
KVL

SEQ ID NO: 114 - protein, 9 aa, Artificial
  synthetic peptide
KVLKVLKVL

SEQ ID NO: 115 - protein, 7 aa, Artificial
  synthetic peptide
KVLALRL

SEQ ID NO: 116 - protein, 15 aa, Artificial
  synthetic peptide
KVLKVLKVLKVLKVL

SEQ ID NO: 117 - protein, 17 aa, Artificial
  synthetic peptide
KVLINWKGIAAMAKKII

SEQ ID NO: 118 - protein, 17 aa, Artificial
  synthetic peptide
RVGINWKGIAAMAKKII

SEQ ID NO: 119 - protein, 3 aa, Artificial
  synthetic peptide
ALR

SEQ ID NO: 120 - protein, 15 aa, Artificial
  synthetic peptide
ALRALRALRALRALR

SEQ ID NO: 121 - protein, 18 aa, Artificial
  synthetic peptide
ALRALRALRALRALRALR

SEQ ID NO: 122 - protein, 18 aa, Artificial
  synthetic peptide
KVLKVLKVLKVLKVLKVL